SEQUENCE LISTING (22 sequences)

SEQ ID NO: 1 - DNA, 20 bp, Artificial Sequence
  Description of Artificial Sequence Synthetic primer
tcgcgaggaccaacccagtc

SEQ ID NO: 2 - DNA, 20 bp, Artificial Sequence
  Description of Artificial Sequence Synthetic primer
ccgctcctacgcccaatcac

SEQ ID NO: 3 - DNA, 20 bp, Artificial Sequence
  Description of Artificial Sequence Synthetic primer
gccacttcaaaagggagatg

SEQ ID NO: 4 - DNA, 20 bp, Artificial Sequence
  Description of Artificial Sequence Synthetic primer
tccatgtgaatccaagttgc

SEQ ID NO: 5 - DNA, 30 bp, Artificial Sequence
  Description of Artificial Sequence Synthetic primer
cagcctcggtaccggcggatctccgaaacc

SEQ ID NO: 6 - DNA, 30 bp, Artificial Sequence
  Description of Artificial Sequence Synthetic primer
gcggacgccgaggctcttgagctcgctgac

SEQ ID NO: 7 - protein, 13 aa, Artificial Sequence
  Description of Artificial Sequence Synthetic peptide
SAGWKNSIRHNLS

SEQ ID NO: 8 - DNA, 22 bp, Artificial Sequence
  Description of Artificial Sequence Synthetic primer
gggcactgtccgagtagccagc

SEQ ID NO: 9 - DNA, 21 bp, Artificial Sequence
  Description of Artificial Sequence Synthetic primer
gcaatactgtaataggaactc

SEQ ID NO: 10 - DNA, 21 bp, Artificial Sequence
  Description of Artificial Sequence Synthetic primer
gagcgtgccctacttcaagga

SEQ ID NO: 11 - protein, 14 aa, Artificial Sequence
  Description of Artificial Sequence Synthetic peptide
GDSNSSAGWKNSIR

SEQ ID NO: 12 - protein, 16 aa, Artificial Sequence
  Description of Artificial Sequence Synthetic peptide
DKGDSNSSAGWKNSIR

SEQ ID NO: 13 - protein, 8 aa, Artificial Sequence
  Description of Artificial Sequence Synthetic peptide
HNLSLHSR

SEQ ID NO: 14 - protein, 12 aa, Artificial Sequence
  Description of Artificial Sequence Synthetic peptide
SSWWIINPDGGK

SEQ ID NO: 15 - protein, 15 aa, Artificial Sequence
  Description of Artificial Sequence Synthetic peptide
SSWWIINPDGGKSGK

SEQ ID NO: 16 - protein, 18 aa, Artificial Sequence
  Description of Artificial Sequence Synthetic peptide
SSWWIINPDGGKSGKAPR

SEQ ID NO: 17 - protein, 32 aa, Homo sapiens
SAGWKNSIRHNLSLHSRFMRVQNEGTGKSSWW

SEQ ID NO: 18 - protein, 32 aa, Mus sp.; identical to SEQ ID NO: 17

SEQ ID NO: 19 - protein, 32 aa, Homo sapiens
SAGWKNSIRHNLSLHSKFIRVQNEGTGKSSWW

SEQ ID NO: 20 - protein, 32 aa, Mus sp.; identical to SEQ ID NO: 19

SEQ ID NO: 21 - protein, 32 aa, Caenorhabditis elegans
SAGWKNSIRHNLSLHSRFMRIQNEGAGKSSWW

SEQ ID NO: 22 - protein, 32 aa, Drosophila sp.
SAGWKNSIRHNLSLHNRFMRVQNEGTGKSSWW

(19) United States
(12) Patent Application Publication
(10) Pub. No.: US 2008/0242608 A1
(43) Pub. Date: Oct. 2, 2008

(54) Methods and compositions for treating and preventing neurologic disorders

(76) Inventors: Azad Bonni, Brookline, MA (US); Maria Lehtinen, Brookline, MA (US); Zengqiang Yuan, Bejing (CN)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/809,565

(22) Filed: May 31, 2007

Related U.S. Application Data
(60) Provisional application No. 60/810,348, filed on Jun. 2, 2006.

(51) Int. Cl. A61K 38/16; G01N 33/53; A61K 31/7105; A61K 38/03

(52) U.S. Cl. 514/12; 514/16; 514/15; 514/14; 514/13; 435/7.1; 514/44

(57) ABSTRACT

The present invention provides methods for treating or reducing neurologic and ischemic vascular disorders.

### RELATED APPLICATION

This application claims priority to U.S. Ser. No. 60/810,348, filed Jun. 2, 2006, which is incorporated herein by reference in its entirety.

### STATEMENT AS TO FEDERALLY SPONSORED RESEARCH

This invention was funded in part by the U.S. Government under grant number NS041021 awarded by the National Institute of Neurological Disorders and Stroke (NINDS). The Government has certain rights in the invention.

### BACKGROUND OF THE INVENTION

Cell death of neurons is a fundamental process in the development of the nervous system and contributes to the pathogenesis of several neurologic disorders. Since neurons are postmitotic cells that last the entire lifespan of an organism, specific mechanisms have evolved to regulate cell death in neurons. Since the mechanisms underlying neuron-specific mechanisms of cell death remains poor, there is a dearth of treatment modalities for neurologic disorders that involve excessive neurodegenerative disorders.

### SUMMARY OF THE INVENTION

The invention provides a method of reducing neural cell death (e.g., apoptosis or necrosis) by contacting a neural cell (e.g., cortical neuron, hippocampal neurons, cerebellar granule neurons, and spinal cord neurons) with an agent that reduces the level or activity of the protein kinase MST (e.g., MST1). Agents such as substrate analogs or products thereof preferentially reduce apoptosis in neural cells compared to non-neural cells. For example, the inhibitor reduces cell death at least 20%, 50%, 100%, 2 fold, 5-fold and up to ten fold in neural cells as compared to non-neural cells. By reducing neural cell death, this agent is useful for treating or preventing neurologic or neurodegenerative disorders including Alzheimer's disease, multiple sclerosis, Parkinson's disease, amyotrophic lateral sclerosis, stroke, cerebral ischemic disease, Huntington's disease, spinal muscular atrophy, stroke, brain trauma, spinal cord injury, and diabetic neuropathy. Additional disorders that may be treated according to the present invention include those that involved oxidative stress, such as myocardial ischemic and peripheral ischemic disease; diabetic retinopathy, and diabetic nephropathy. For example, the agent reduces cell death by reducing the ability of MST1 to bind and phosphorylate a FOXO transcription factor (e.g., FOXO3). Exemplary agents are small molecule inhibitors and RNA agents.

A small molecule inhibitor is a compound that is less than 2000 daltons in mass. The molecular mass of the inhibitory compounds is preferably less than 1000 daltons, more preferably less than 600 daltons, e.g., the compound is less than 500 daltons, 400 daltons, 300 daltons, 200 daltons, or 100 daltons. Preferably, the inhibitor is not a peptide or proteinaceous in nature.

Peptide agents are also useful. For example, the peptide is at least 8, 10, 20, 30, 40 residues in length and reduces phosphorylation of FOXO3 by MST1.

The invention also provides methods for identifying a candidate compound for reducing or preventing death in a neural cell. These methods involve the steps of: (a) contacting a cell expressing an MST1 gene with a candidate compound; and (b) measuring MST1 gene expression or protein activity in the cell. A candidate compound that reduces the expression or the activity of MST1 relative to such expression or activity in a cell that has not been contacted with the candidate compound is useful for reducing or preventing neural cell apoptosis. For example, the candidate compound reduces the ability of MST1 to bind and phosphorylate FOXO3. Optionally, the MST1 gene is an MST1 fusion gene and the MST1 expressing cell is a mammalian cell (e.g., a rodent or human cell). In other embodiments, step (b) involves the measurement of the level of MST1 mRNA or protein.

Alternatively, the method involves the steps of: (a) contacting an MST1 protein with a candidate compound; and (b) determining whether the candidate compound binds the MST1 protein and/or reduces MST1 activity. Candidate compounds that bind and reduce MST1 activity are identified as compounds useful for reducing or preventing neural cell death. Preferably, the candidate compound reduces the ability of MST1 to phosphorylate a FOXO transcription factor.

In yet another screening approach, a method for identifying a candidate compound for reducing or preventing neural cell death involves the steps of: (a) contacting an MST1 protein (e.g., human MST1 protein) with a candidate compound; and (b) determining whether the candidate compound reduces binding of MST1 to a FOXO transcription factor. The candidate compound is first contacted with MST1, a FOXO transcription factor, or is simultaneously contacted with both proteins or fragments thereof, e.g., a fragment of MST1. Candidate compounds that reduce such binding reduce or prevent neural cell apoptosis. Other screening methods involve screening for compounds that reduce the ability of FOXO3 to translocate from the cytoplasm to the nucleus in neural cells. Another screening approach involves screening for compounds that reduce the ability of FOXO3 to induce the expression of cell death genes. Optionally, such neural cells are exposed to an agent that induces death or that activates MST1.

In all foregoing aspects of the invention, candidate compounds identified as being useful for reducing or preventing neural cell apoptosis are useful to treat or prevent neural disorders.

By “reduce the expression or activity of MST1” is meant to reduce the level or biological activity of MST1 relative to the level or biological activity of MST1 in an untreated control. The level or activity is preferably reduced by at least 10%, 20%, 30%, 40%, 50%, 60%, 70%, 80%, 90%, or 100%, relative to an untreated control. Since MST1 phosphorylates FOXO transcription factors, a reduction in the biological activity of MST1 is, for example, a reduction in the phosphorylation or activity level of FOXO transcription factors, in turn resulting in a reduction in apoptosis. For example, the phosphorylation of FOXO transcription factors is reduced by at least 10%, 20%, 30%, 40%, 50%, 60%, 70%, 80%, 90%, or even 100% relative to an untreated control, thereby reducing apoptosis and ultimately treating or reducing neural or neurodegenerative disorders. Thus, as used herein, the term “activity” with respect to an MST1 polypeptide includes any activity which is inherent to the naturally occurring MST1 protein, such as binding and phosphorylation of FOXO transcription factors, activation of neural apoptosis, or both, as detected by any standard method.

By “treating or preventing a neurologic disorder” is meant ameliorating any of the conditions or symptoms associated with the disorder before or after it has occurred including, for example, seizures, headaches, and memory loss. Alternatively, alleviating a symptom of a disorder may involve reducing visible areas of neuronal cell death relative to an untreated control. As compared with an equivalent untreated control, such reduction or degree of prevention is at least 5%, 10%, 20%, 40%, 50%, 60%, 80%, 90%, 95%, or 100% as measured by any standard technique. A patient who is being treated for a neurologic disorder is one who a medical practitioner has diagnosed as having such a condition. Diagnosis may be by any suitable means. Diagnosis and monitoring may involve, for example, detecting the presence of destroyed or dying neurons in a biological sample (e.g., tissue biopsy, blood test, or urine test), detecting the presence of amyloid plaques, detecting the level of a surrogate marker of the neurologic disorder in a biological sample, abnormal MRI or other imaging diagnosis, or detecting symptoms associated with the neurologic disorder. A patient in whom the development of a neurologic disorder is being prevented may or may not have received such a diagnosis. If the latter situation, the subject has one or more risk factors for the disorder, e.g., a family history or aberrant gene expression profile associated with the disorder. One in the art will understand that these patients may have been subjected to the same standard tests as described above or may have been identified, without examination, as one at high risk due to the presence of one or more risk factors (e.g., family history or genetic predisposition).

As used herein, by “MST1” is meant a polypeptide that phosphorylates a FOXO transcription factor and is involved in various signaling pathways including oxidative stress-induced apoptosis. The MST1 proteins of the invention are substantially identical to the naturally occurring MST1 (e.g., accession numbers NM_{—}006282 and NP_{—}006273 (both human) as well as NM_{—}021420 and NP_{—}067395 (both murine), the sequences of which are hereby incorporated by reference). Neurologic disorders are treated or prevented when MST1 activity or expression is increased by at least 5%, 10%, 20%, 30%, 40%, 50%, 60%, 70%, 80%, 90%, or 100% above control levels as measured by any standard method (e.g., Northern blot analysis).

By an “MST1 gene” is meant a nucleic acid that encodes an MST1 protein.

By “MST1 fusion gene” is meant an MST1 promoter and/or all or part of an MST1 coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and beta-galactosidase.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 20 amino acids, preferably at least 30 amino acids, more preferably at least 40 amino acids, and most preferably 50 amino acids or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

The term “isolated DNA” is meant DNA that is free of the genes which, in the naturally occurring genome of the organism from which the given DNA is derived, flank the DNA. Thus, the term “isolated DNA” encompasses, for example, cDNA, cloned genomic DNA, and synthetic DNA.

By “an effective amount” is meant an amount of a compound, alone or in a combination, required to reduce or prevent the neurologic disorder in a mammal. The effective amount of active compound(s) varies depending upon the route of administration, age, body weight, and general health of the subject. Ultimately, the attending physician or veterinarian will decide the appropriate amount and dosage regimen.

By a “candidate compound” is meant an agent to be evaluated as a MST1 inhibitor. Candidate compounds may include, for example, peptides, polypeptides, synthetic organic molecules, naturally occurring organic molecules, nucleic acid molecules, peptide nucleic acid molecules, and components and derivatives thereof.

The term “pharmaceutical composition” is meant any composition, which contains at least one therapeutically or biologically active agent and is suitable for administration to the patient. For example, the composition includes the active agent in a pharmaceutically acceptable excipient. Any of these formulations can be prepared by well-known and accepted methods of the art. See, for example, Remington: The Science and Practice of Pharmacy, 20^{th} edition, (ed. A. R. Gennaro), Mack Publishing Co., Easton, Pa., 2000.

The invention provides significant advantages over standard therapies for treatment, prevention, and reduction, or alternatively, the alleviation of one or more symptoms associated with neurologic disorders, because it preferentially targets neural cells compared to non-neural cells, thereby reducing or eliminating adverse side effects associated with therapeutic agents that are less or not cell type specific. In addition, the screening methods allow for the identification of therapeutics that modify the injury process rather than merely mitigating the symptoms.

Cited publications including sequences defined by GENBANK™ accession numbers are incorporated herein by reference.

Other features, objects, and advantages of the invention will be apparent from the description of the drawings.

### BRIEF DESCRIPTION OF THE FIGURES

FIG. 1A is a series of immunoblot photographs. Lysates of granule neurons treated with increasing concentrations of H₂O₂ (0-100 μM) for 30 minutes were immunoblotted with the phospho-MST1/2 and MST1 antibodies.

FIG. 1B is a series of photographs showing an immunocytochemical analysis of granule neurons transfected with GFP-MST1 and β-galactosidase expression plasmids together with the MST1 RNAi or control U6 plasmid. MST1 RNAi reduced GFP-MST1 expression in 91.3% of transfected cells (t-test, p<0.0001, n=3).

FIG. 1C is a bar graph. Granule neurons were transfected with the MST1 RNAi or control U6 plasmid together with the β-galactosidase expression plasmid. Percent cell death in transfected β-galactosidase-positive neurons is represented as mean ±SEM. Cell death was significantly increased upon H₂O₂ treatment in U6-transfected neurons but not in MST1 knockdown neurons (ANOVA, p<0.05, n=4). In these and other survival experiments, H₂O₂ treatment similarly induced cell death in untransfected neurons in cultures transfected with different test plasmids.

FIG. 1D is a series of immunoblot photographs. Lysates of 293T cells transfected with an expression vector encoding FLAG-MST1 or FLAG-MST1R together with the MST1 RNAi or control U6 plasmid were immunoblotted with the FLAG and ERK1/2 antibodies.

FIG. 1E is a bar graph. Neurons transfected with the FLAG-MST 1 or FLAG-MST1R expression plasmid and the β-galactosidase expression vector together with the MST1 RNAi plasmid were treated with H₂O₂ and analyzed as in FIG. 1C. MST1R blocked the ability of MST1 RNAi to protect neurons from H₂O₂-induced death (ANOVA, p<0.001, n=3).

FIG. 1F is a bar graph. Neurons transfected with the FOXO RNAi, Cdk2 RNAi, or control U6 plasmid together with the β-galactosidase expression vector were left untreated or treated with H₂O₂ and analyzed as in FIG. 1C. FOXO knockdown protected neurons from H₂O₂-induced death (ANOVA, p<0.0001, n=3).

FIG. 1G is a bar graph. Neurons were transfected with the FLAG-MST1 and the β-galactosidase expression vectors together with the FOXO RNAi or control U6 plasmid. After 72 hours, cultures were analyzed as in FIG. 1C. FOXO knockdown blocked MST1-induced neuronal death (ANOVA, p<0.05, n=3).

FIG. 2A is a series of immunoblot photographs. Lysates of 293T cells transfected with an expression vector encoding FLAG-MST1, kinase-dead FLAG-MST1 K59R, or the control plasmid were immunoprecipitated with the FLAG antibody and subjected to an in vitro kinase assay using full-length His-FOXO3 as substrate in the presence of ³²P-ATP. Auto-p denotes autophosphorylated MST1. Lower panel shows the expression of MST1 and MST1 K59R by immunoblotting.

FIG. 2B is a series of immunoblot photographs. Lysates of 293T cells transfected with the GFP-FOXO3 and FLAG-MST1 expression plasmids were immunoprecipitated with the GFP or control HA (ctrl) antibody followed by immunoblotting with the FLAG antibody.

FIG. 2C is a series of immublot photographs. Lysates of 293T cells were immunoprecipitated with the control HA (ctrl) or a rabbit antibody to FOXO3 followed by immunoblotting with an antibody to MST1 or MLK3.

FIG. 2D is a series of immublot photographs. GST-pulldown assays of FLAG-MST1 with recombinant GST or GST fused with fragments of FOXO3 (P1 to P5). Precipitated proteins were immunoblotted with the FLAG antibody. Lower panel is Coomassie Blue (CB) staining of GST-FOXO3 peptides.

FIG. 2E is a series of immunoblot photographs showing representing in vitro kinase assays using FLAG-MST1 with no substrate, recombinant GST, or recombinant GST-FOXO3 peptides were done as in FIG. 2A.

FIG. 2F is an alignment of sequences in the forkhead domain of mammalian FOXO3 and FOXO1, C. elegans DAF-16, and Drosophila dFOXO.

FIG. 2G is a series of immunoblot photographs. Lysates of 293T cells transfected with FLAG-MST1, FLAG-MST1 K59R, or the control vector were immunoprecipitated with the FLAG antibody and subjected to an in vitro kinase assay using recombinant His-FOXO3 as substrate. Phosphorylation reactions were analyzed by immunoblotting with the phospho-FOXO, His, or FLAG antibody.

FIG. 2H is a series of immunoblot photographs. Lysates of 293T cells transfected with the FLAG-MST1 expression vector or the control plasmid together with expression plasmids encoding GFP-FOXO3 or GFP-FOXO3 mutants were subjected to immunoblotting with the phospho-FOXO, GFP, or FLAG antibody.

FIG. 3A is a series of immunoblot photographs. Lysates of granule neurons left untreated or treated with H₂O₂ for indicated times were subjected to immunoblotting with the phospho-FOXO, FOXO3, or β-Actin antibody.

FIG. 3B is a series of photographs showing immufluorescent stains. Neurons left untreated or treated with H₂O₂ for one hour were subjected to immunocytochemical analysis with the phospho-FOXO antibody. The kinetics of H₂O₂-induced endogenous FOXO3 phosphorylation (sustained at 1 hour, declined at three hours, and was very low at 24 hours) preceded cell death.

FIG. 3C is a series of immunoblot photographs. Lysates of 293T cells (left panel) or primary neurons (right panel) left untreated or treated with H₂O₂ were immunoprecipitated with the FOXO3 or control HA (ctrl) antibody followed by immunoblotting with the MST1 antibody.

FIG. 3D is a series of immunoblot photographs. Lysates of 293T cells transfected with the MST1 RNAi or control U6 plasmid and then treated with H₂O₂ for 1 hour were immunoblotted with the phospho-FOXO, MST1, or FOXO3 antibody.

FIG. 3E is a is a series of photographs and a bar graph showing an immunocytochemical analysis of granule neurons that were transfected with the MST1, MST2, or Cdk2 RNAi plasmid or the control U6 plasmid together with the β-galactosidase expression vector and then treated with H₂O₂ for one hour. MST1 and MST2 knockdown inhibited H₂O₂-induced FOXO phosphorylation as compared to control (ANOVA, p<0.01, n=3).

FIG. 4A is a series of immunoblot photographs. Lysates of 293T cells transfected with the GFP-FOXO3 expression plasmid together with an expression vector encoding FLAG-MST1, FLAG-MST1 K59R, or the control vector were immunoprecipitated with the GFP antibody followed by immunoblotting with an antibody against 14-3-3β.

FIG. 4B is a series of immunoblot photographs. Lysates of 293T cells transfected with expression plasmids encoding wild type or mutant GFP-FOXO3 together with the FLAG-MST1 expression plasmid or its control vector were immunoprecipitated with the GFP antibody and immunoblotted with the 14-3-3β antibody.

FIG. 4C is a series of immunoblot photographs. Lysates of 293T cells left untreated or treated with H₂O₂ for 1 hour were immunoprecipitated with the FOXO3 antibody followed by immunoblotting with the 14-3-3β antibody.

FIG. 4D is a series of immunoblot photographs. In the left panel, lysates of 293T cells transfected with the MST1 and MST2 RNAi plasmids or control U6 plasmid were immunoblotted with the MST1, MST2, or ERK1/2 antibodies. Asterisks indicate non-specific bands. In the right panel, lysates of 293T cells transfected with both the MST1 and MST2 RNAi plasmids or the control U6 plasmid together with the GFP-FOXO3 expression plasmid and then left untreated or treated with H₂O₂ were subjected to immunoprecipitation analysis as in FIG. 4A.

FIG. 4E is a bar graph. CCL39 cells transfected with expression plasmids encoding wild type GFP-FOXO3 or its mutants together with the FLAG-MST1 expression plasmid or its control vector were subjected to immunocytochemical analysis with the GFP antibody and the DNA dye bisbenzimide (Hoechst 33258). MST1 induced nuclear localization of wild type GFP-FOXO3 (ANOVA, p<0.01, n=3) but not of the GFP-FOXO3 mutants. A minimum of 200 cells were counted per condition.

FIG. 4F is a series of photographs of immunofluorescent stains and a bar graph. Neurons transfected with GFP-FOXO3 or GFP-FOXO3 S207A were left untreated or treated with H₂O₂ for one hour and subjected to immunocytochemical analysis using confocal microscopy. H₂O₂ induced the nuclear translocation of GFP-FOXO3 but not of GFP-FOXO3 S207A. Quantitation (average of 2 independent experiments) is shown.

FIG. 5A is a series of immunoblot photographs. Lysates of neurons treated with increasing concentrations of H₂O₂ (0-100 μM) for 18 hours were immunoblotted with antibodies to BIM and Hsp60.

FIG. 5B is a series of immunoblot photographs. Lysates of 293T cells transfected with the MST1 RNAi or control U6 plasmid and then treated with increasing concentrations of H₂O₂ (0-50 μM) for 12 hours were subjected to immunoblotting with the BIM, MST1, or 14-3-3β antibody.

FIG. 5C is a bar graph. Granule neurons were transfected with the MST1 or FOXO RNAi plasmid or control U6 plasmid together with the BIM-luciferase reporter gene and the tk-renilla reporter, the latter to serve as an internal control for transfection efficiency. After 72 hours, cells were treated with H₂O₂ for 12 hours and subjected to luciferase assays. Shown are mean ±SEM of normalized firefly/renilla luciferase values relative to the control U6 transfected neurons. MST1 and FOXO knockdown significantly reduced H₂O₂-induced BIM-luciferase reporter gene expression (ANOVA, p<0.01, n=3).

FIG. 5D is a series of immunoblot photographs. Lysates of 293T cells transfected with the FOXO RNAi or control U6 plasmid and a GFP-FOXO3 or GFP-FOXO3R expression vector were immunoblotted with the GFP or ERK1/2 antibodies. Asterisk denotes nonspecific signal. Expression of FOXO hpRNAs induced knockdown of FOXO3 but not FOXO3R.

FIG. 5E is a bar graph and a series of immunoblot photographs. In the left panel, neurons transfected with the FOXO RNAi plasmid together with expression vectors encoding FOXO3, FOXO3R, FOXO3R S207A, FOXO3R 4A or their control vector and β-galactosidase were treated with H₂O₂ and analyzed as in FIG. 1D. FOXO3R but not mutants of FOXO3R in which serine 207 was replaced with alanine blocked the ability of FOXO knockdown to protect neurons from H₂O₂-induced death (ANOVA, p<0.0001, n=3). In the right panel, lysates of 293T cells transfected with FOXO3, FOXO3R, FOXO3R S207A, FOXO3R 4A or their control vector were immunoblotted with the FOXO3 or ERK1/2 antibodies.

FIG. 6A is a series of immunoblot photographs. In vitro kinase assays were carried out by incubating recombinant MST1 (Upstate) together with recombinant His-DAF-16 followed by immunoblotting with the phospho-FOXO or His antibody.

FIG. 6B is a series of immunoblot photographs. Lysates of 293T cells transfected with an expression plasmid encoding HA-DAF-16 or HA-DAF-16 S196A together with the FLAG-MST1 expression vector or control plasmid were immunoblotted with the phospho-FOXO, HA, or FLAG antibody.

FIG. 6C is a series of photographs showing Pcst1::gfp expression in wild type nematodes. Fluorescent (a) and Nomarski (b) views of an L4 nematode are shown and are representative of more than five independent transgenic lines at all postembryonic stages examined (L1-adult).

FIG. 6D is a bar graph (left panel) showing CST knockdown shortens nematode lifespan. For statistical analysis, see Table 2. The right panel is a series of photographs showing an RT-PCR from synchronized L4 nematodes maintained on bacteria harboring the cst-1 RNAi plasmid or control bacteria for 2 generations.

FIGS. 6E and 6F are graphs showing that CST knockdown reduced body movement when compared to control (log rank, p<0.0001; animals observed/initial number examined: N2=42/53, cst-1[RNAi]=42/50). CST knockdown also reduced pharyngeal pumping when compared to control (log rank, p<0.01; animals observed/number examined: N2=39/53, cst-1 [RNAi]=38/50).

FIG. 6G is a series of photographs showing CST knockdown in Pmyo-3::myo-3::gfp nematodes promoted advanced muscle deterioration, including sarcomere disorganization, bending, and fragmentation. Images are representative of day 14 adult nematodes.

FIG. 6H is a bar graph and series of photographs showing that age-associated oily droplets between the pharyngeal bulbs developed earlier in response to CST knockdown when compared to control N2 nematodes (days 5 and 6; ANOVA, p<0.05). Increased cst-1 expression delayed the onset and progression of oily droplets (day 7; ANOVA, p<0.01). Arrowheads refer to representative oily droplets. The right panel is a series of photographs of an RT-PCR showing increased cst-1 gene dosage in Ex1050 young adult nematodes.

FIG. 6I is a graph. C. elegans movement was scored by their ability to move during a 20 second time interval in response to gentle tapping of plates. Increased gene dosage of cst-1 delays the onset of age-related physiological processes (log rank, p<0.01; n: Ex1060=26/50, Ex1050=39/55).

FIG. 7A is a graph showing that lifespan extension induced by overexpression of cst-1 is daf-16-dependent. Ex1050 nematodes were fed bacteria containing the daf-16 RNAi plasmid. For statistical analyses, see Table 2.

FIG. 7B is a graph showing that CST knockdown does not influence lifespan of daf-16 (mgDf47) nematodes.

FIG. 7C is a graph showing that overexpression of cst-1 increases DAF-2 knockdown nematode lifespan.

FIGS. 7D and 7E are graphs showing that CST knockdown reduces lifespan in daf-2 (e1368) and age-1 (hx546) nematodes to a similar extent as in control N2 nematodes.

FIG. 7F is a diagram showing a model of the MST-FOXO signaling pathway.

FIG. 8 is a series of immunoblot photographs. The phospho-FOXO antibody recognizes recombinant MST1-phosphorylated FOXO3 at serine 207 in vitro. In vitro kinase assays were carried out by incubating purified recombinant MST1 (Upstate) with recombinant GST-FOXO3 forkhead domain followed by immunoblotting with the phospho-FOXO, GST, or MST1 antibody.

FIG. 9 is a series of immunoblot photographs. Hydrogen peroxide induces the phosphorylation of endogenous FOXO3 at serine 207 in 293T cells. Lysates of 293T cells transfected with the FOXO RNAi or control U6 plasmid and then left untreated or treated with H₂O₂ for 1 hour were immunoblotted with the phospho-FOXO, FOXO3, or β-Actin antibody.

FIG. 10 is a series of photographs of immunofluorescent stains and a bar graph. MST1, but not MST1K59R, induces nuclear accumulation of FOXO3. CCL39 cells transfected with the GFP-FOXO3 expression plasmid together with an expression plasmid encoding FLAG-MST1, FLAG-MST1 K59R, or the control vector were subjected to immunocytochemical analysis with the GFP antibody and the DNA dye bisbenzimide (Hoechst 33258). Quantitation revealed that the percentage of cells with nuclear localization of GFP-FOXO3 was significantly increased upon expression of MST1 but not MST1 K59R as compared to control vector (ANOVA, p<0.01, n=3). A minimum of 200 cells per condition were counted.

FIG. 11 is a PCR gel photograph and an immunoblot photograph. Mutation of serine 207 does not appear to reduce FOXO3 binding to DNA. Crosslinked chromatin was prepared from 293T cells transfected with GFP-FOXO3 or GFP-FOXO3 S207A expression plasmid using a lipofectamine method (to obtain high level of expression to override normal controls of FOXO subcellular localization) and immunoprecipitated with the FOXO3 antibody or beads alone (ctrl) followed by PCR amplification using primer pairs specific for the BIM promoter. Input reflects DNA extracts from chromatin before immunoprecipitation. Mutation of serine 207 did not appear to reduce the ability of FOXO3 to bind the promoter of the BIM gene. Immunoblot (bottom panel) shows equal expression of wild type and S207A mutant FOXO3. Wild type or S207A mutant FOXO3 expressed at high levels in 293T cells by lipofectamine (and then treated with H₂O₂) were also found to similarly bind to the BIM promoter in vitro in gel mobility shift assays.

FIGS. 12A and 12B is a diagram showing that C. elegans contains two genes, cst-1 and cst-2, with homology to MST1. FIG. 12A shows that the cst-1 gene is predicted to produce two transcripts encoding proteins that differ by 2 amino acids (http://www.wormbase.org, release WS144). Open boxes indicate exons containing coding sequence. Arrows indicate the predicted direction of transcription. FIG. 12B shows an alignment of the predicted proteins encoded by cst-1b and cst-2. The cst-1 RNAi construct has 100% sequence identity with the corresponding region in cst-2, and therefore will induce knockdown of both cst-1 and cst-2.

FIG. 13 is a bar graph. Increased cst-1 gene dosage induces the DAF-16 target gene hsp-12.6. Total RNA was isolated with Trizol from synchronized young adult C. elegans, reverse transcribed with Superscript II, and analyzed by real-time PCR. The ratio of hsp-12.6/spt-4 is shown. Overexpression of cst-1 induced hsp-12.6 expression (t-test, p<0.01, n=5).

### DETAILED DESCRIPTION OF THE INVENTION

The invention is based on the discovery that the protein kinase MST1 plays a key role in neural apoptosis associated with oxidative stress and other stressors.

Oxidative stress influences cell survival and homeostasis. Our results demonstrate that the protein kinase MST 1 mediates oxidative stress-induced cell death in primary mammalian neurons by directly activating the FOXO transcription factors. MST1 phosphorylates FOXO proteins at a conserved site within the forkhead domain that disrupts their interaction with 14-3-3 proteins, promotes FOXO nuclear translocation, and thereby induces cell death in neurons. We also extend the MST-FOXO signaling link to nematodes. Knockdown of the C. elegans MST1 ortholog CST-1 shortens lifespan and accelerates tissue aging, while overexpression of CST-1 promotes lifespan and delays aging. The CST-1-induced lifespan extension occurs in a DAF-16-dependent manner. The identification of the FOXO transcription factors as major and evolutionarily conserved targets of MST1 suggests that MST kinases play important roles in diverse biological processes including cellular responses to oxidative stress and longevity.

The experiments described herein were performed using the following Materials and Methods.

### Plasmids

Fragments of GST-FOXO3 and GST-FOXO1-forkhead domain plasmids were cloned by PCR into pGEX4T1 at the EcoRI and XhoI restriction sites. His-FOXO3 was cloned into pET3a vector at the EcoRI and BamHI restriction sites. FOXO3 S207A and −4A, and FOXO1 S212A, and −4A were generated by site-directed mutagenesis. All mutations were verified by sequencing.

### Antibodies

Antibodies to MST1 (Zymed); phospho-MST1 (Thr183)/MST2 (Thr180), MST2, ERK1/2 (Cell Signaling); FOXO3 (Upstate); GFP (Molecular Probes); GST, MLK3, His, HA, 14-3-3β, Hsp60, β-Actin (Santa Cruz Biotechnology); FLAG-M2 (Sigma); BIM (Stressgen) were purchased. The rabbit antibody to phosphorylated serine 207 of FOXO3 was generated by injecting New Zealand rabbits with the phosphopeptide antigen C-SAGWKNpSIRHNLS and was purified as described (Konishi et al., 2002).

### Tissue Culture

Cerebellar granule neuron cultures were prepared from postnatal day 6 rat pups as described (Konishi et al., 2002). For RNAi experiments, cultures from P6+2DIV were transfected with the RNAi or control U6 plasmid, together with a plasmid encoding β-galactosidase. After 3 days, cultures were left untreated or treated with H₂O₂ (60-100 μM; Fisher) for 24 hours, fixed and subjected to cell survival as described (Konishi et al., 2002). Cell counts were carried out in a blinded manner and analyzed for statistical significance by ANOVA, followed by Fisher's PLSD post-hoc test. Approximately 150 cells were counted per experiment. Unless stated otherwise, all transfections were done by a calcium-phosphate method as described (Konishi et al., 2002).

### Immunoprecipitation, Immunoblotting, and Kinase Assays

In vitro kinase assays were carried out as described (Graves et al., 2001). Immunoprecipitations and immunoblotting were carried out as described (Konishi et al., 2002).

### Mass Spectrometry

Coomassie Blue-stained bands corresponding to FOXO3 and FOXO1 (unphosphorylated or phosphorylated with MST1) were digested with trypsin as described (Peng and Gygi, 2001). Peptide mixtures were separated by reverse-phase chromatography and online analyzed on a hybrid linear ion trap-ion cyclotron resonance Fourier transform instrument (LTQ-FT, Thermo Finnigan) using a TOP10 method. MS3 scans were triggered only for doubly-charged ions demonstrating an intense neutral loss of phosphoric acid (Beausoleil et al., 2004). Spectra were searched using Sequest algorithm. Peptide matches obtained were deemed correct after applying several filtering criteria (tryptic ends, XCorr >1.8 and 2.7 for 2+ and 3+ ions, respectively; mass error <5 ppm) and manual validation.

### RNAi Plasmid Design

Mammalian RNAi constructs were designed as described (Gaudilliere et al., 2002). The hpRNA targeting sequences used include: MST1 hpRNA: G GGC ACT GTC CGA GTA GCC AGC; MST2 hpRNA: GC AAT ACT GTA ATA GGA ACT C, and FOXO hpRNA: G AGC GTG CCC TAC TTC AAG GA. MST1 Rescue and FOXO Rescue were generated by creating five silent base pair mutations into the wild type cDNA encoding MST1 or FOXO3 using the Quick Change Site-Directed Mutagenesis Kit (Stratagene). For cst-1 RNAi experiments, a cDNA fragment of cst-1 (nucleotides 121-1141) was generated by PCR from cDNA clone yk103e9.

Extrachromosomal Transgenic C. elegans

To examine the expression pattern of cst-1, ˜1.7 kb of the predicted promoter region was cloned into the GFP expression vector pPD95.75 (Pcst1::gfp). The Pcst1::gfp plasmid (50 ng/ul) was injected together with the pRF4 [rol-6 (su1006)] plasmid (100 ng/ul), used as the transformation marker in all experiments. To express CST-1, the endogenous locus including ˜1.7 kb of upstream regulatory sequence and the entire coding and 3′ UTR regions was cloned into pBs (pBs-cst-1) and injected at 2.5 ng/μl.

C. elegans Lifespan Analysis

Lifespan experiments were carried out as described (An and Blackwell, 2003). Each experiment was carried out over several plates such that for an experiment with n=100, 4 plates containing 25 nematodes were used. During lifespan analysis, C. elegans were observed daily for movements. If no movement was detected, nematodes were prodded gently with a platinum wire and examined for pharyngeal pumping to determine if alive. Worms that escaped from the plates or exploded were censored. Statistical analysis (log rank, Mantel-Cox) was carried out using JMP-IN 5.1 statistical software. Maximal lifespan of Ex1050 and N2 nematodes in each experiment was calculated by obtaining the mean ±SEM of maximum lifespan of all plates in each experiment and subjected to statistical analysis using the t-test.

### Chromatin Immunoprecipitation

1×10⁷ 293T cells transfected with FOXO3 or FOXO3 S207A were used per experiment. Cells were crosslinked with 0.75% formaldehyde for 10 min., harvested, and sonicated in the ChIP lysis buffer (1% Triton X-100, 1 mM EDTA, 50 mM Tris-HCl, 500 mM NaCl, 0.1% Na-Deoxycholate, 0.1% SDS and protease inhibitors) to produce soluble chromatin with an average size of 300-1000 bp. Polyclonal anti-FOXO3 antibody (3 μg) (Upstate) was added to each sample and incubated overnight at 4° C. To collect the immunocomplex, 30 μl of salmon protein-A agarose beads were added to the samples and incubated for 1 hr at 4° C. The beads were washed with lysis buffer, wash buffer (0.1% Triton X-100, 5 mM EDTA, 30 mM Tris-HCl, 150 mM NaCl), TE buffer, and in 50 mM Tris/10 mM EDTA. The bound protein-DNA immunocomplexes were eluted with 100 μl elution buffer (50 mM Tris pH 8.0, 10 mM EDTA/1% SDS) and de-crosslinked at 65° C. for 4 hr. Next, 250 μl TE, 5 μg Glucogen Blue, 100 μg Proteinase K were added to the eluates and incubated at 37° C. for 2 hours. The de-crosslinked chromatin DNA was further purified by QIAquick PCR Purification Kit (Qiagen) and eluted in 50 μl TE buffer. Two μl of eluted DNA sample was used for each PCR reaction. Thirty six PCR cycles were used for FOXO3 ChIP. Primers used for amplifications were as follows:

(SEQ ID NO: 1)

BIM (forward): 5′-TCG CGA GGACCA ACC CAG TC-3′

(SEQ ID NO: 2)

BIM (reverse): 5′-CCG CTC CTA CGC CCA ATC AC-3′.

### Real-Time PCR

Total worm RNA was isolated by Trizol (Invitrogen) from synchronized young adult N2 or Excst-1 (01) nematodes and was reverse transcribed by extension of oligodT primers using Superscript II (Invitrogen). Real-time PCR was performed using Lightcycler Faststart DNA Master SYBR Green I (Roche) with the following pairs of primers:

HSP-12.6 (forward):

(SEQ ID NO: 3)

5′-GCC ACT TCA AAA GGG AGA TG-3′
HSP-12.6 (reverse):

(SEQ ID NO: 4)

5′-TCC ATG TGA ATC CAA GTT GC-3′
SPT-4 (forward):

(SEQ ID NO: 5)

5′-CAG CCT CGG TAC CGG CGG ATC TCC GAA ACC-3′
SPT-4 (reverse):

(SEQ ID NO: 6)

5′-GCG GAC GCC GAG GCT CTT GAG CTC GCT GAC-3′

### Therapeutic Agents

An inhibitor of MST1 is any agent having the ability to reduce the expression or the activity of MST1 in a cell. The inhibitor preferentially inhibits neural cell death. The control cell is a cell that has not been treated with the MST1 activator. MST1 expression or activity is determined by any standard method in the art, including those described herein. MST1 inhibitors include polypeptides, polynucleotides, small molecule antagonists, or siRNA. For example, a MST1 inhibitor reduces MST1 activity by reducing binding between MST1 and FOXO transcription factors (e.g., FOXO3).

Alternatively, the MST1 inhibitor is a dominant negative protein or a nucleic acid encoding a dominant negative protein that interferes with the biological activity of MST1. A dominant negative protein is any amino acid molecule having a sequence that has at least 50%, 70%, 80%, 90%, 95%, or even 99% sequence identity to at least 10, 20, 35, 50, 100, or more than 150 amino acids of the wild type protein to which the dominant negative protein corresponds. For example, a dominant-negative MST1 has mutation such that it no longer activates downstream pathways. Specifically, a dominant-negative MST1 binds FOXO transcription factors (e.g., FOXO3) less efficiently than the naturally-occurring MST1 polypeptide and therefore fails to activate apoptosis.

The dominant negative protein may be administered as a nucleic acid in an expression vector. The expression vector may be a non-viral vector or a viral vector (e.g., recombinant retrovirus, recombinant lentivirus, recombinant adeno-associated virus, or a recombinant adenoviral vector). Alternatively, the dominant negative protein may be directly administered as a recombinant protein systemically or to the affected area using, for example, microinjection techniques.

The MST1 inhibitor is an antisense molecule, an RNA interference (siRNA) molecule, a small molecule antagonist that targets MST1 expression or activity, or a vector that directs production of such inhibitory compositions. By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense MST1 nucleic acid sequence, an anti-sense MST1 nucleic acid sequence or both. Optionally, the siRNA is constructed such that a single transcript has both the sense and complementary antisense sequences from the target gene, e.g., a hairpin. Binding of the siRNA to a MST1 transcript in the target cell results in a reduction in MST1 production by the cell. The length of the oligonucleotide is at least 10 nucleotides and may be as long as the naturally-occurring MST1 transcript. Preferably, the oligonucleotide is 19-25 nucleotides in length. Most preferably, the oligonucleotide is less than 75, 50, 25 nucleotides in length.

Small molecules includes, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Useful small molecules may reduce MST1 expression or activity by reducing the interaction between MST1 and FOXO transcription factors. Inhibitors include small molecules and peptides.

A biologically active dose of a MST1 inhibitor is a dose that will reduce neural apoptosis. Desirably, the MST1 inhibitor has the ability to reduce the expression or activity of MST1 in neuronal cells (e.g., granule neurons) by at least 5%, 10%, 20%, 30%, 40%, 50%, 60%, 70%, 80%, 90%, or 100% below untreated control levels. The levels or activity of MST1 in cells is measured by any method known in the art, including, for example, Western blot analysis, immunohistochemistry, ELISA, and Northern Blot analysis. Alternatively, the biological activity of MST1 is measured by assessing the expression or activity of any of the molecules involved in MST1 signaling. The biological activity of MST1 is determined according to its ability to reduce neural cell apoptosis. Preferably, the agent that reduces the expression or activity of MST1 can reduce neural cell apoptosis or neurodegeneration by at least 5%, 10%, 20%, 30%, 40%, 50%, 60%, 70%, 80%, 90%, or 100% below untreated control levels. The agent of the present invention is therefore any agent having any one or more of these activities.

Optionally, the subject is administered one or more additional therapeutic regiments. The additional therapeutic regimens may be administered prior to, concomitantly, or subsequent to administration of the MST1 inhibitor. For example, the MST1 inhibitor and the additional agent are administered in separate formulations within at least 1, 2, 4, 6, 10, 12, 18, or more than 24 hours apart. Optionally, the additional agent is formulated together with the MST1 inhibitor. When the additional agent is present in a different composition, different routes of administration may be used. The agent is administered at doses known to be effective for such agent for treating, reducing, or preventing the progression of the neural disorder.

Concentrations of the MST1 inhibitor and the additional agent depends upon different factors, including means of administration, target site, physiological state of the mammal, and other medication administered. Thus treatment dosages may be titrated to optimize safety and efficacy and is within the skill of an artisan. Determination of the proper dosage and administration regime for a particular situation is within the skill of the art.

MST1 inhibitors are administered in an amount sufficient to reduce neural apoptosis or neurodegeneration. Such reduction includes the alleviation of one or more of symptoms associated with the neural disorder being treated or prevented. Administration of the MST1 inhibitor reduces the neurodegeneration associated with the neural disorder or alleviates one or more symptoms associated with the disorder by at least 10%, 20%, 30%, 40%, 50%, 60%, 70%, 80%, 90%, or 100% as compared to an untreated subject.

Treatment is efficacious if the treatment leads to clinical benefit such as, a reduction of the symptoms of a neurologic disorder in the subject. When treatment is applied prophylactically, “efficacious” means that the treatment retards or prevents the neurodegenerative process. Efficacy may be determined using any known method for diagnosing or treating the neural disorder.

### Therapeutic Administration

The invention includes administering to a subject a composition that includes a compound that reduces MST1 expression or activity (referred to herein as an “MST1 inhibitor” or “therapeutic compound”). As described herein, this inhibitor may reduce binding between MST1 and FOXO transcription factors (e.g., FOXO3).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other agents or therapeutic agents for treating, preventing or alleviating a symptom of a neurodegenerative disorder. A therapeutic regimen is carried out by identifying a mammal, e.g., a human patient suffering from (or at risk of developing) a neural disorder, using standard methods.

The pharmaceutical compound is administered to such an individual using methods known in the art. Preferably, the compound is administered orally, rectally, nasally, topically or parenterally, e.g., subcutaneously, intraperitoneally, intrathecally, intramuscularly, and intravenously. The compound is administered prophylactically, or after the detection of the neurologic injury. Compounds are also delivered locally to neural tissue (e.g., brain, spinal cord, or peripheral neural tissues) make direct contact with a site of injury or disease. The compound is optionally formulated as a component of a cocktail of therapeutic drugs to treat the neural disorder. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the MST1 inhibitor is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

Where the therapeutic compound is a nucleic acid encoding a protein, the therapeutic nucleic acid is administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. A nucleic acid therapeutic is introduced intracellularly and incorporated within host cell DNA or remain episomal.

For local administration of DNA, standard gene therapy vectors used. Such vectors include viral vectors, including those derived from replication-defective hepatitis viruses (e.g., HBV and HCV), retroviruses (see, e.g., WO 89/07136; Rosenberg et al., 1990, N. Eng. J. Med. 323(9):570-578), adenovirus (see, e.g., Morsey et al., 1993, J. Cell. Biochem., Supp. 17E,), adeno-associated virus (Kotin et al., 1990, Proc. Natl. Acad. Sci. USA 87:2211-2215,), replication defective herpes simplex viruses (HSV; Lu et al., 1992, Abstract, page 66, Abstracts of the Meeting on Gene Therapy, September 22-26, Cold Spring Harbor Laboratory, Cold Spring Harbor, N.Y.), and any modified versions of these vectors. The invention may utilize any other delivery system which accomplishes in vivo transfer of nucleic acids into eucaryotic cells. For example, the nucleic acids may be packaged into liposomes, e.g., cationic liposomes (Lipofectin), receptor-mediated delivery systems, non-viral nucleic acid-based vectors, erythrocyte ghosts, or microspheres (e.g., microparticles; see, e.g., U.S. Pat. No. 4,789,734; U.S. Pat. No. 4,925,673; U.S. Pat. No. 3,625,214; Gregoriadis, 1979, Drug Carriers in Biology and Medicine, pp. 287-341 (Academic Press,). Naked DNA may also be administered.

DNA for gene therapy can be administered to patients parenterally, e.g., intravenously, subcutaneously, intramuscularly, and intraperitoneally. DNA or an inducing agent is administered in a pharmaceutically acceptable carrier, i.e., a biologically compatible vehicle which is suitable for administration to an animal e.g., physiological saline. A therapeutically effective amount is an amount which is capable of producing a medically desirable result, e.g., a decrease of a MST1 gene product in a treated animal. Such an amount can be determined by one of ordinary skill in the art. As is well known in the medical arts, dosage for any given patient depends upon many factors, including the patient's size, body surface area, age, the particular compound to be administered, sex, time and route of administration, general health, and other drugs being administered concurrently. Dosages may vary, but a preferred dosage for intravenous administration of DNA is approximately 10⁶ to 10²² copies of the DNA molecule. Typically, plasmids are administered to a mammal in an amount of about 1 nanogram to about 5000 micrograms of DNA. Desirably, compositions contain about 5 nanograms to 1000 micrograms of DNA, 10 nanograms to 800 micrograms of DNA, 0.1 micrograms to 500 micrograms of DNA, 1 microgram to 350 micrograms of DNA, 25 micrograms to 250 micrograms of DNA, or 100 micrograms to 200 micrograms of DNA. Alternatively, administration of recombinant adenoviral vectors encoding the MST1 inhibitor into a mammal may be administered at a concentration of at least 10⁵, 10⁶, 10⁷, 10⁸, 10⁹, 10¹⁰, or 10¹¹ plaque forming unit (pfu).

MST1 gene products are administered to the patient intravenously in a pharmaceutically acceptable carrier such as physiological saline. Standard methods for intracellular delivery of peptides can be used, e.g. packaged in liposomes. Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, and intraperitoneal.

MST1 inhibitors are effective upon direct contact of the compound with the affected tissue or may alternatively be administered systemically (e.g., intravenously, rectally or orally). The MST1 inhibitor may be administered intravenously or intrathecally (i.e., by direct infusion into the cerebrospinal fluid). For local administration, a compound-impregnated wafer or resorbable sponge is placed in direct contact with CNS tissue. The compound or mixture of compounds is slowly released in vivo by diffusion of the drug from the wafer and erosion of the polymer matrix. Alternatively, the compound is infused into the brain or cerebrospinal fluid using standard methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (described, for example, in U.S. Pat. No. 5,954,687) provides a fluid flow path suitable for the transfer of fluids to or from selected location at, near or within the brain to allow administration of the drug over a period of time.

Patients treated according to the invention may have been subjected to the tests to diagnose a subject as having a neurologic disorder or may have been identified, without examination, as one at high risk due to the presence of one or more risk factors (e.g., genetic predisposition). Reduction of neurodegenerative symptoms or damage may also include, but are not limited to, alleviation of symptoms (e.g., headaches, nausea, skin rash), diminishment of extent of disease, stabilized (i.e., not worsening) state of disease, delay or slowing of disease progression, and amelioration or palliation of the disease state. Treatment may occur at home with close supervision by the health care provider, or may occur in a health care facility.

### Screening Assays

The present invention provides screening methods to identify compounds that can inhibit the expression or activity of MST1. Useful compounds include any agent that inhibits the biological activity or reduces the cellular level of MST1. For example, useful compounds are identified by detecting an attenuation of the expression or activity of any of the molecules involved in MST1 signaling. For example, a useful compound reduces binding between MST1 and FOXO transcription factors. The screening assays may also identify agents that reduce neural cell apoptosis.

A number of methods are available for carrying out such screening assays. According to one approach, candidate compounds are added at varying concentrations to the culture medium of cells expressing MST1. Gene expression of MST1 is then measured, for example, by standard Northern blot analysis, using any appropriate fragment prepared from the nucleic acid molecule of MST1 as a hybridization probe or by real time PCR with appropriate primers. The level of gene expression in the presence of the candidate compound is compared to the level measured in a control culture medium lacking the candidate molecule. If desired, the effect of candidate compounds may, in the alternative, be measured at the level of MST1 polypeptide using the same general approach and standard immunological techniques, such as Western blotting or immunoprecipitation with an antibody specific to MST1 for example. For example, immunoassays may be used to detect or monitor the level of MST1. Polyclonal or monoclonal antibodies which are capable of binding to MST1 may be used in any standard immunoassay format (e.g., ELISA or RIA assay) to measure the levels of MST1. MST1 can also be measured using mass spectroscopy, high performance liquid chromatography, spectrophotometric or fluorometric techniques, or combinations thereof.

As a specific example, mammalian cells (e.g., rodent cells) that express a nucleic acid encoding MST1 are cultured in the presence of a candidate compound (e.g., a peptide, polypeptide, synthetic organic molecule, naturally occurring organic molecule, nucleic acid molecule, or component thereof). Cells may either endogenously express MST1 or may alternatively be genetically engineered by any standard technique known in the art (e.g., transfection and viral infection) to overexpress MST1. The expression level of MST1 is measured in these cells by means of Western blot analysis and subsequently compared to the level of expression of the same protein in control cells that have not been contacted by the candidate compound. A compound which promotes a decrease in the level of MST1 activity as a result of reducing its synthesis or biological activity is considered useful in the invention.

Alternatively, the screening methods of the invention may be used to identify candidate compounds that decrease the biological activity of MST1 by reducing neural cell apoptosis by at least 10%, 20%, 30%, 40%, 50%, 60%, 70%, 80%, 90%, 95%, or 100% relative to an untreated control. As another alternative, candidate compounds are identified for their ability to reduce binding between MST1 and FOXO transcription factors. A candidate compound may be tested for its ability to reduce such binding in neural cells that naturally express MST1 and FOXO transcription factors (e.g., FOXO3) or after transfection with cDNA for MST1 and FOXO transcription factors, or in cell-free solutions containing MST1 and FOXO transcription factors, as described further below. The effect of a candidate compound on the binding or activation of FOXO transcription factors can be tested by radioactive and non-radiaoctive binding assays, competition assays, and receptor signaling assays.

Given its ability to decrease the biological activity of MST1, such a molecule may be used, for example, as a therapeutic agent to treat, reduce, or prevent a neural disorder, or alternatively, to alleviate one or more symptoms associated with such a disorder. As a specific example, a candidate compound may be contacted with two proteins, the first protein being a polypeptide substantially identical to MST1 and the second protein being FOXO transcription factors (e.g., FOXO3) (i.e., a protein that binds the MST1 polypeptide under conditions that allow binding). According to this particular screening method, the interaction between these two proteins is measured following the addition of a candidate compound. A decrease in the binding of MST1 to FOXO transcription factors following the addition of the candidate compound (relative to such binding in the absence of the compound) identifies the candidate compound as having the ability to inhibit the interaction between the two proteins, and thereby having the ability to reduce MST1 activity. The screening assay of the invention may be carried out, for example, in a cell-free system or using a yeast two-hybrid system. If desired, one of the proteins or the candidate compound may be immobilized on a support as described above or may have a detectable group.

Alternatively, or in addition, candidate compounds may be screened for those which specifically bind to and thereby inhibit MST1. The efficacy of such a candidate compound is dependent upon its ability to interact with MST1. Such an interaction can be readily assayed using any number of standard binding techniques and functional assays. For example, a candidate compound may be tested in vitro for interaction and binding with MST1 and its ability to modulate neural cell apoptosis may be assayed by any standard assays (e.g., those described herein).

For example, a candidate compound that binds to MST1 may be identified using a chromatography-based technique. For example, a recombinant MST1 may be purified by standard techniques from cells engineered to express MST1 and may be immobilized on a column. Alternatively, the naturally-occurring MST1 may be immobilized on a column. A solution of candidate compounds is then passed through the column, and a compound specific for MST1 is identified on the basis of its ability to bind to MST1 and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography).

Screening for new inhibitors and optimization of lead compounds may be assessed, for example, by assessing their ability to modulate MST1 activity using standard techniques. Compounds which are identified as binding to MST1 with an affinity constant less than or equal to 10 mM are considered particularly useful in the invention.

Potential therapeutic agents include organic molecules, peptides, peptide mimetics, polypeptides, and antibodies that bind to a nucleic acid sequence encodes MST1 or a MST1 peptide and thereby inhibit or extinguish their activity. Inhibitory agents also include small molecules that bind to and occupy domains of MST1 or FOXO transcription factors (e.g., FOXO3) that interact with each other. Additional inhibitors include agents that inhibit the autophosphorylation of MST (e.g., MST1). Other potential agents include antisense molecules.

This invention is based in part on the experiments described in the following examples. These examples are provided to illustrate the invention and should not be construed as limiting.

### MST1 Mediates Oxidative Stress-Induced Cell Death Via FOXO Transcription Factors

Primary granule neurons of the rat cerebellum provide a robust system for the study of cell death including upon exposure to oxidative stress (Becker and Bonni, 2004).

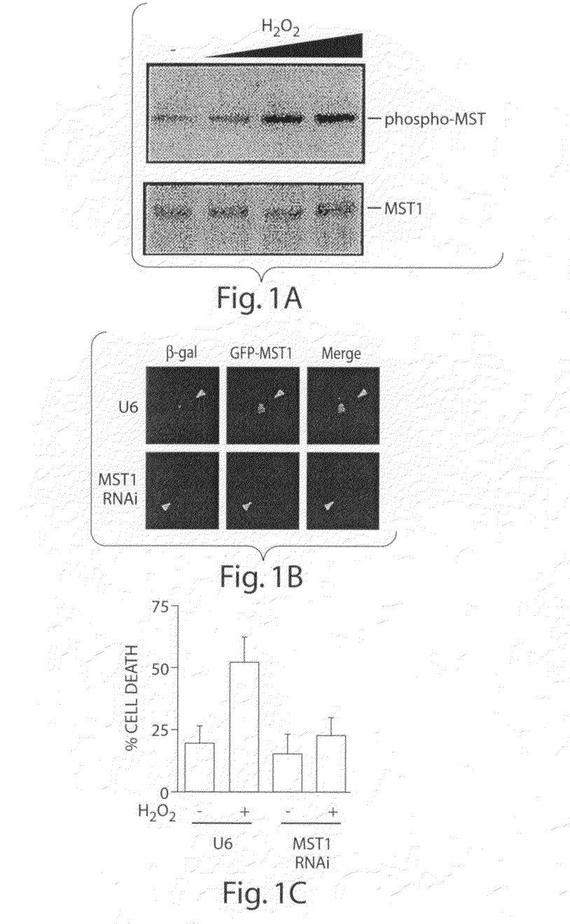

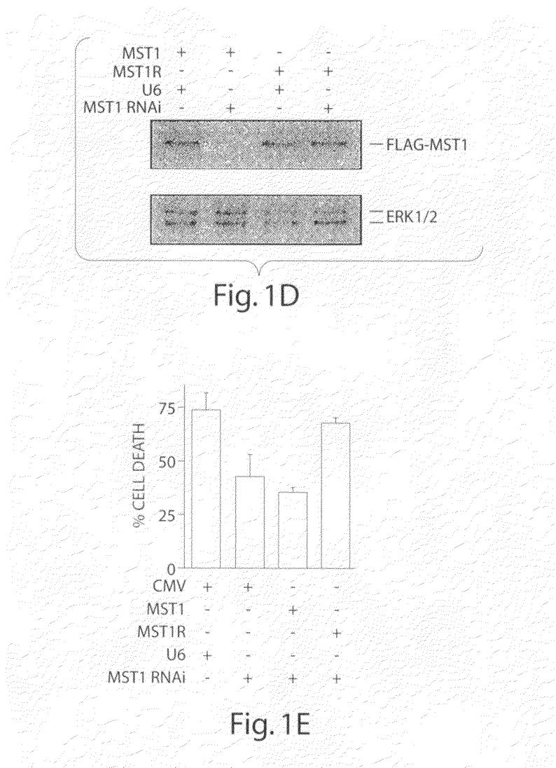

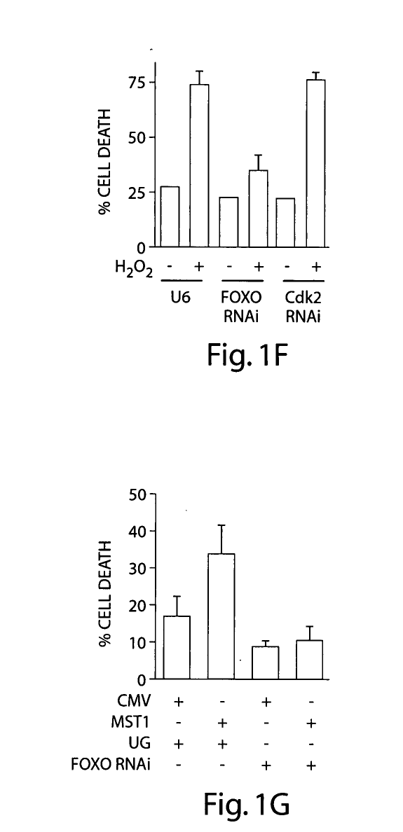

The exposure of granule neurons to hydrogen peroxide stimulated the increased autophosphorylation of MST1/2 (FIG. 1A), indicating the MST family of kinases is activated in neurons in response to oxidative stress.

To determine the importance of oxidative stress-induced endogenous MST1 in neurons, a plasmid-based method of RNA interference (RNAi) was employed. Expression of MST1 hairpin RNAs (hpRNAs) reduced effectively the expression of MST1 in granule neurons (FIG. 1B). Primary neurons were transfected with the MST1 RNAi or control U6 plasmid, and three days after transfection neurons were left untreated or treated with hydrogen peroxide for 24 hours. Exposure of control U6-transfected neurons to hydrogen peroxide robustly induced cell death. However, MST1 knockdown neurons were protected from hydrogen peroxide-induced cell death (FIG. 1C). These data indicate that MST1 is required for oxidative stress-induced neuronal cell death.

To demonstrate the specificity the MST1 RNAi phenotype in neurons, we performed a rescue experiment. We generated a “rescue” form of MST1 (MST1R) that is resistant to MST1 RNAi (FIG. 1D). MST1R, but not MST1, significantly reduced the ability of MST1 knockdown to protect neurons from hydrogen peroxide-induced cell death (FIG. 1E). Thus, MST1 RNAi inhibits hydrogen peroxide-induced cell death via specific knockdown of MST1 rather than off-target effects of RNAi.

The mechanism by which MST1 promotes neuronal cell death was next investigated. In S. cerevisiae, hydrogen peroxide triggers the translocation of full length Ste20 to the nucleus, where Ste20 phosphorylates histone H₂B at serine 10 (Ahn et al., 2005). In mammalian cells, the cytotoxic agent etoposide induces the cleavage of an N-terminal fragment of MST1 that localizes to the nucleus and phosphorylates histone H₂B at serine 14 (Cheung et al., 2003). Our findings that hydrogen peroxide does not induce the nuclear translocation of MST1 or the phosphorylation of histone H₂B at serine 14 in mammalian cells and neurons, raised the possibility that MST1 might couple oxidative stress signals to the nucleus via proteins that undergo nucleocytoplasmic shuttling.

The FOXO proteins transit between the cytoplasm and nucleus and mediate responses to oxidative stress including neuronal death (Brunet et al., 2004; Essers et al., 2004). Whether MST1 mediates oxidative stress-induced cell death in neurons via FOXO proteins was next addressed. Induction of FOXO RNAi, but not of the unrelated protein Cdk2, protected neurons from hydrogen peroxide-induced cell death (FIG. 1F), indicating a requirement for FOXO family proteins in oxidative stress-induced neuronal cell death. In other experiments, expression of exogenous MST1 in neurons induced cell death, but knockdown of FOXO proteins suppressed MST1-induced cell death (FIG. 1G). Together, these results indicate that MST1 mediates oxidative stress-induced cell death in a FOXO-dependent manner.

### MST1 Phosphorylates FOXO3 at a Conserved Site In Vitro and In Vivo

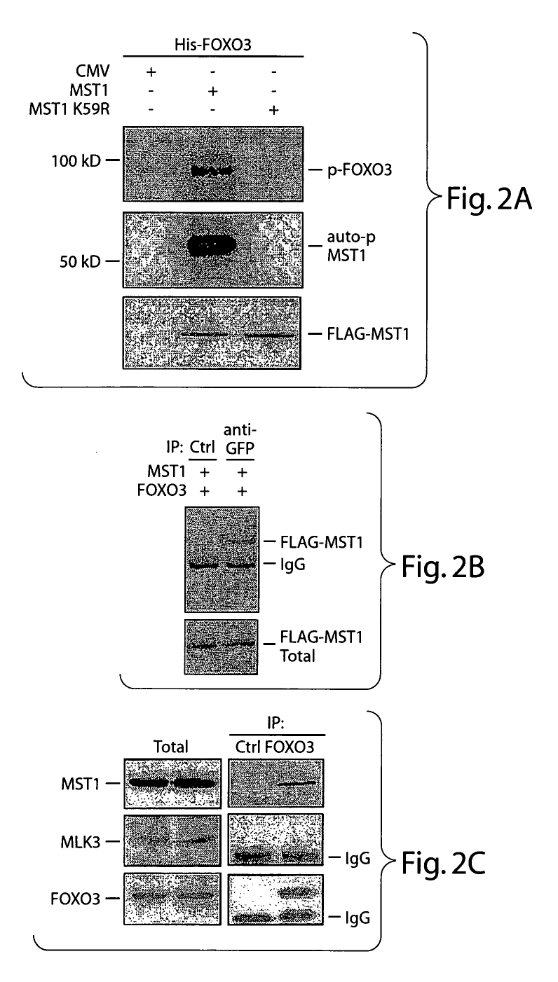

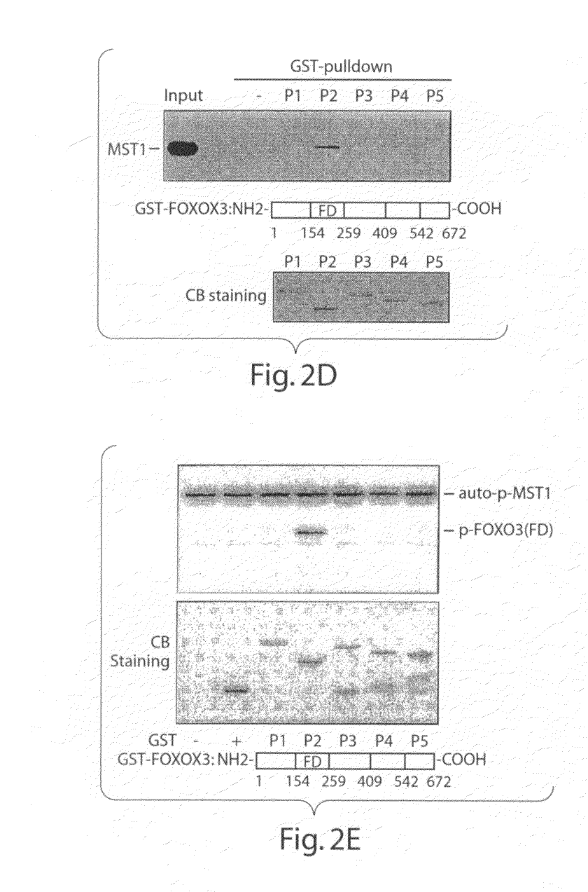

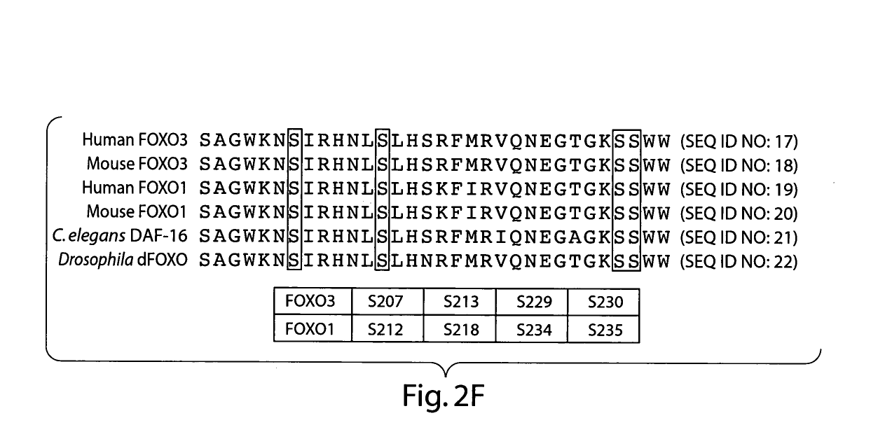

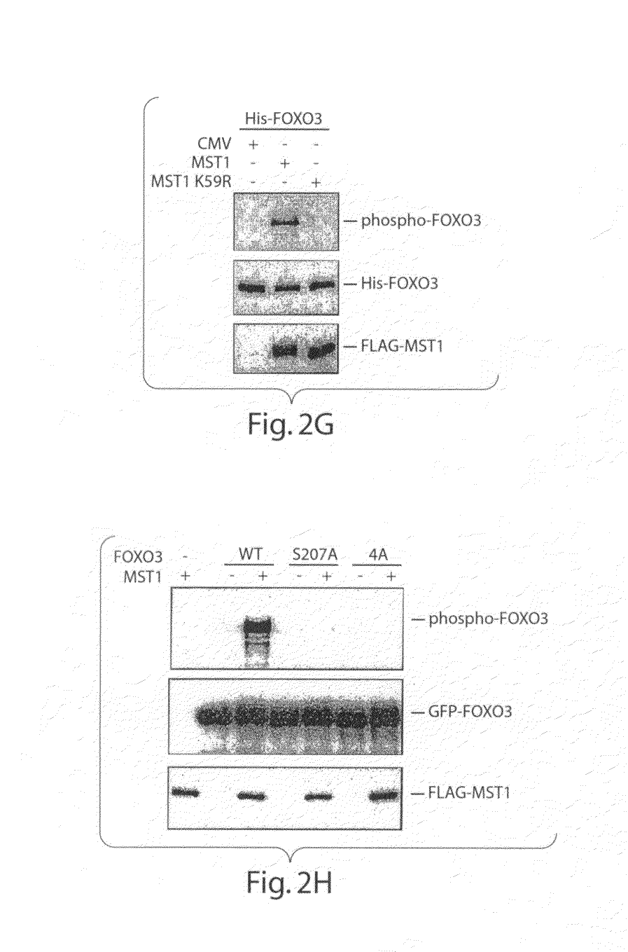

Since MST1 is a protein kinase, whether MST1 phosphorylates FOXO proteins was next examined. MST1, but not a kinase-dead MST1 in which the ATP binding site was mutated (MST1 K59R), phosphorylated FOXO3 in vitro (FIG. 2A). Upon expression in 293T cells, MST1 and FOXO3 formed a physical complex (FIG. 2B). Endogenous MST1, but not the protein kinase MLK3, associated with endogenous FOXO3 in cells (FIG. 2C). The interaction of MST1 and FOXO3 indicate that FOXO3 is a substrate of MST1.

The region within FOXO3 that associates with MST1 in GST-pull down assays was delineated using recombinant GST fusion proteins encoding five non-overlapping FOXO3 regions (peptides P1-P5). MST1 coprecipitated only with peptide P2 (amino acids 154-259), which contains the forkhead domain (FIG. 2D). MST1 robustly phosphorylated the P2 fragment but not the other FOXO3 fragments in vitro (FIG. 2E). These results indicate that MST1 specifically interacts with and phosphorylates the forkhead domain of FOXO3.

By tandem mass spectrometry analysis (MS/MS) aided by data-dependent MS³, we identified four serine residues (serines 207, 213, 229 or 230, and 241) that were phosphorylated by MST1 in the forkhead domain of FOXO3 (Table 1). These serines are highly conserved among FOXO family members and across species including vertebrates, C. elegans, and Drosophila (FIG. 2F). MST1 phosphorylated the conserved sites in the FOXO1 forkhead domain in vitro, but failed to phosphorylate FOXO 1 forkhead domain mutants in which serine 212 (corresponding to serine 207 in FOXO3) was replaced with alanine. Together, these experiments demonstrate that MST1 phosphorylates the forkhead domain of FOXO transcription factors on distinct sites in vitro, and suggest that one of these sites (serine 207 in FOXO3) represents the principal MST1 phosphorylation site.

TABLE 1
Phosphopeptides detected in the mass spectrometry analysis of FOXO3, after
MST1 kinase in vitro treatment. Mass accuracies from the precursor and Sequest
database searching scores (Xcorr and dCn) for the MS² and MS³ spectra are listed.
(S* denotes phosphoserine).

| | | | mass error | MS² | | MS³ | |
|---|---|---|---|---|---|---|---|
| peptide sequence | site | charge | (ppm) | XCorr | dCn | XCorr | dCn |
| GDSNSSAGWKNS*IR | 207 | 2 | 0.2 | 2.82 | 0.298 | 2.92 | 0.166 |
| DKGDSNSSAGWKNS*IR | 207 | 2 | 1.1 | 2.27 | 0.209 | 4.08 | 0.037 |
| HNLS*LHSR | 213 | 2 | 0.7 | 2.19 | 0.401 | 2.26 | 0.273 |
| (SS)*WWIINPDGGK | 229 or | 2 | 4.5 | 2.46 | 0.005 | 2.66 | 0.001 |
| | 230 | | | | | | |
| SSWWIINPDGGKS*GK | 241 | 2 | 1.2 | 3.91 | 0.478 | 3.66 | 0.460 |
| SSWWIINPDGGKS*GKAPR | 241 | 3 | 3.5 | 3.20 | 0.388 | — | — |

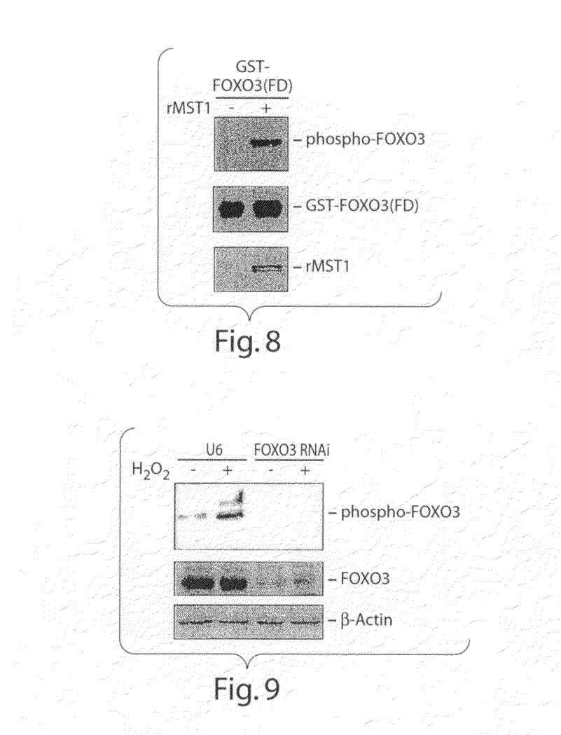

A rabbit antiserum was raised to specifically recognize FOXO proteins when phosphorylated at the conserved MST1 site. The phospho-FOXO antibody recognized recombinant FOXO3 that was phosphorylated by MST1 in vitro, but did not recognize recombinant FOXO3 that was unphosphorylated or that was incubated with the kinase-dead MST1 K59R (FIG. 2G). The phospho-FOXO antibody also specifically recognized the forkhead domain of FOXO3 (FOXO3-FD) that was phosphorylated by purified recombinant MST1 in vitro (FIG. 8). The phospho-FOXO antibody also recognized GFP-FOXO3 but not the serine 207 mutants of GFP-FOXO3 that were coexpressed with MST1 in cells (FIG. 2H). Taken together, our results show that MST1 induces the phosphorylation of FOXO3 at the conserved site within the forkhead domain, serine 207, both in vitro and in vivo.

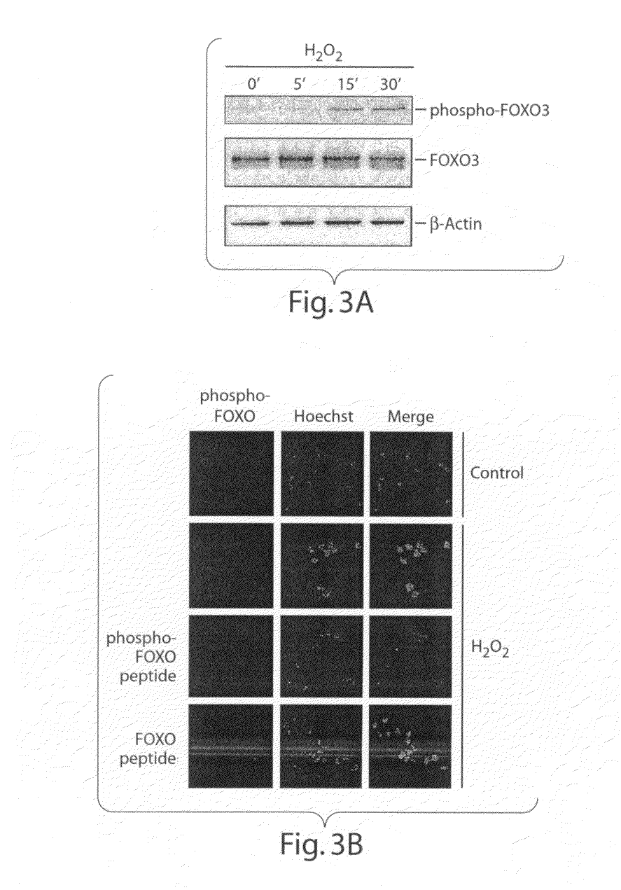

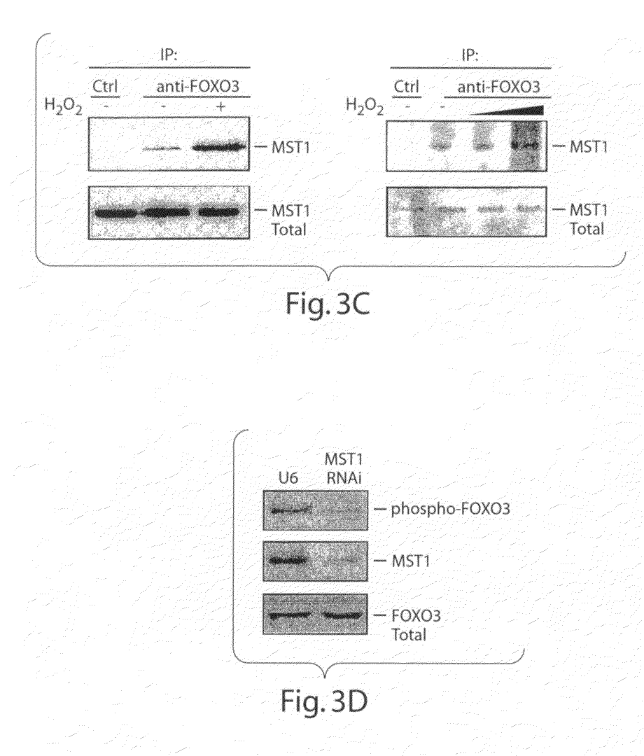

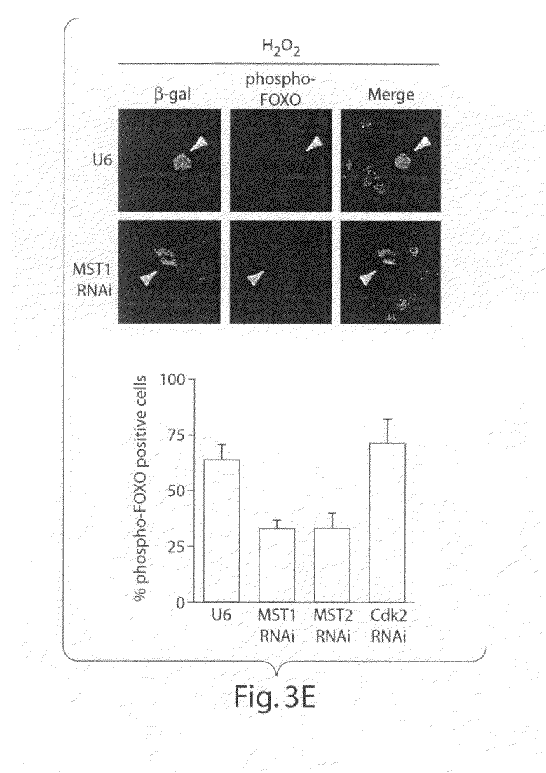

Whether oxidative stress induces the MST1-mediated FOXO phosphorylation in cells and neurons was next determined. Hydrogen peroxide induced the endogenous FOXO3 phosphorylation at serine 207 in both 293T cells (FIG. 9) and primary neurons (FIGS. 3A and B). In the immunocytochemical analyses in neurons, the phospho-FOXO signal was specifically competed with the phosphorylated but not unphosphorylated FOXO peptide (FIG. 3B). Hydrogen peroxide also stimulated the interaction of endogenous MST1 and FOXO3 in cells and primary neurons (FIG. 3C), supporting the possibility that FOXO3 might be a key substrate of MST1 in response to oxidative stress. Knockdown of MST1 in cells impaired hydrogen peroxide-induced phosphorylation of endogenous FOXO3 at serine 207 (FIG. 3D). In neurons, knockdown of MST1 or MST2, but not of the protein kinase Cdk2, reduced the ability of hydrogen peroxide to effectively induce the FOXO3 phosphorylation (FIG. 3E). Taken together, these results indicate that the MST family of kinases phosphorylates FOXO3 at the conserved forkhead domain site, serine 207, in cells and primary neurons upon oxidative stress.

MST1 Phosphorylation of FOXO3 Disrupts its Interaction with 14-3-3 Proteins and Promotes FOXO3 Translocation to the Nucleus

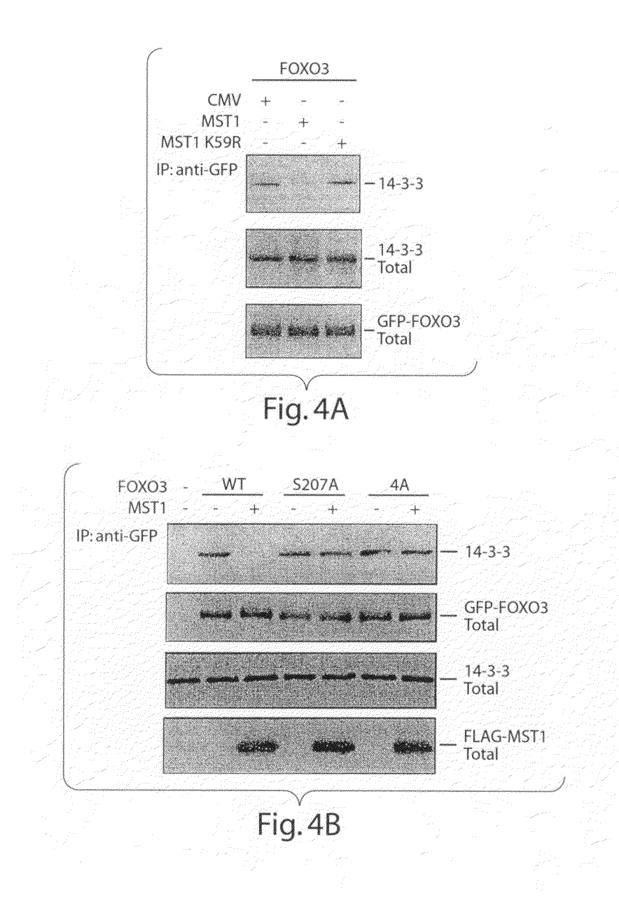

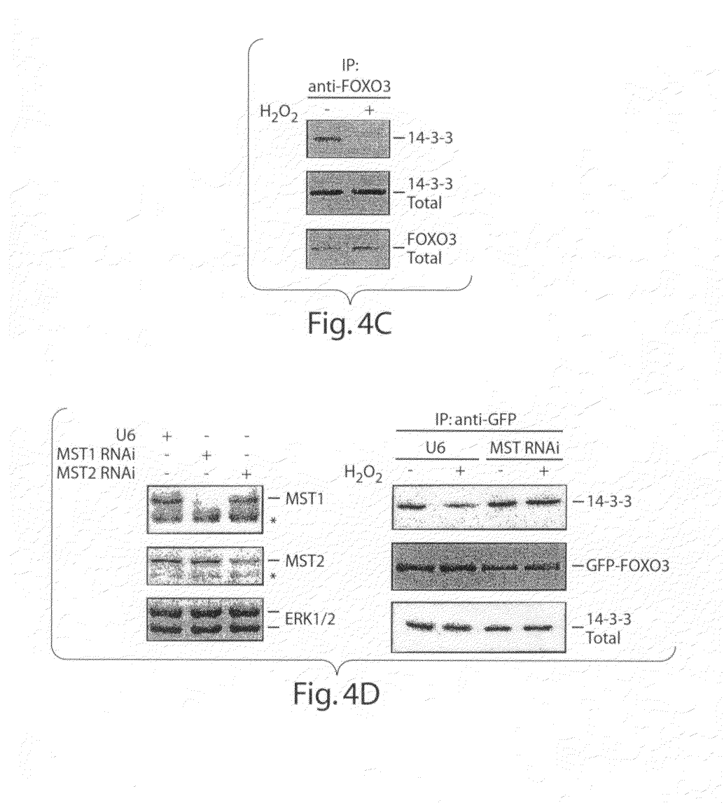

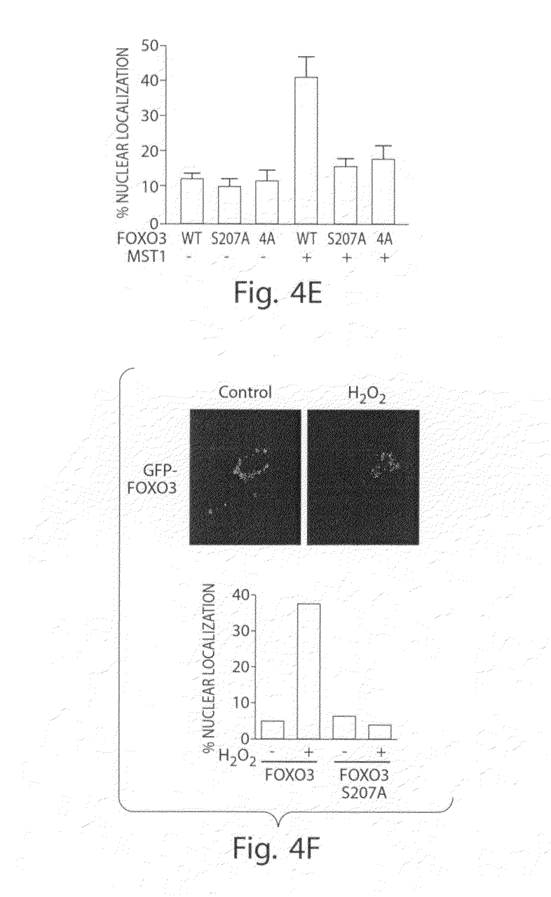

Based on the finding that hydrogen peroxide induces MST1 phosphorylation of FOXO3 at serine 207, the consequences of this phosphorylation event were next determined. Since MST1 interacts with FOXO3 in the cytoplasmic compartment of the cells, whether MST1-induced phosphorylation of FOXO3 regulates FOXO3 's sequestration by 14-3-3 proteins in the cytoplasm. GFP-FOXO3 was expressed together with MST1 or the kinase-dead MST1 K59R in 293T cells. Expression of MST1, but not MST1 K59R, reduced the amount of 14-3-3 that interacted with GFP-FOXO3 (FIG. 4A). These results indicate that MST1 disrupts the association of FOXO3 with 14-3-3 proteins.

The role of the MST1-induced FOXO3 phosphorylation at serine 207 in the inhibition of FOXO3's interaction with 14-3-3 proteins was next determined. While expression of MST1 robustly disrupted the interaction of 14-3-3 proteins with GFP-FOXO3, MST1 failed to inhibit the interaction of 14-3-3 with GFP-FOXO3 mutants in which serine 207 was replaced with alanine (FIG. 4B). MST1 failed to inhibit the phosphorylation of FOXO3 at serine 253, an Akt-induced phosphorylation event that promotes FOXO3's interaction with 14-3-3 proteins (Brunet et al., 2001). These results indicate that MST1-induced phosphorylation of FOXO3 at serine 207 triggers the dissociation of FOXO3 from 14-3-3 proteins.

The role of endogenous MST in the control of FOXO3's interaction with 14-3-3 proteins in response to oxidative stress was determined. Exposure of 293T cells to hydrogen peroxide led to a significant reduction in the association of 14-3-3 proteins with endogenous FOXO3 or exogenously expressed GFP-FOXO3 (FIGS. 4C and 4D). Although induction of MST1 RNAi alone had little effect, knockdown of both MST1 and MST2 together blocked the ability of hydrogen peroxide to inhibit the interaction of 14-3-3 with GFP-FOXO3 in 293T cells (FIG. 4D). Thus, endogenous MST mediates the ability of oxidative stress to trigger the dissociation of FOXO3 and 14-3-3 proteins in cells.

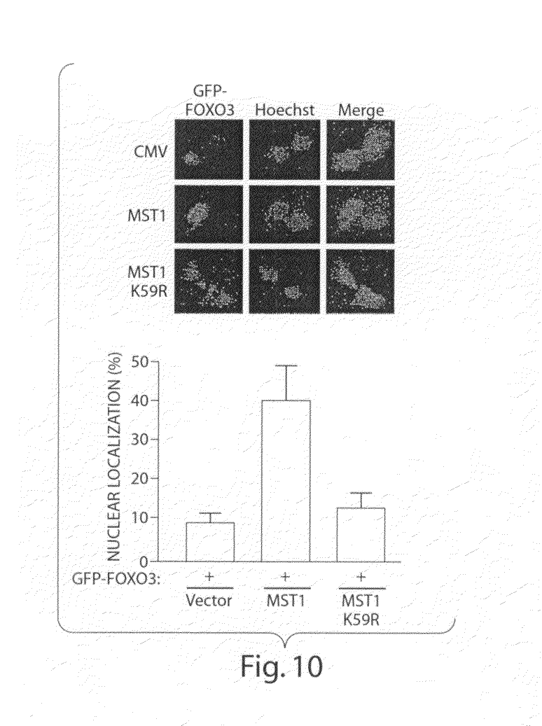

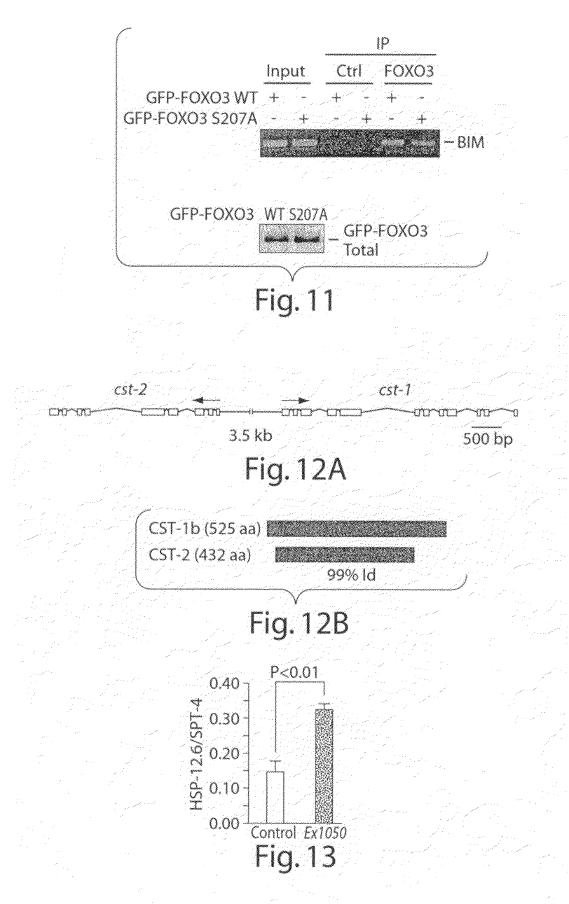

Since 14-3-3 proteins sequester FOXO transcription factors in the cytoplasm (Van Der Heide et al., 2004), the ability of MST-induced disruption of FOXO3-14-3-3 binding to influence the localization of FOXO3 was determined. To measure the effect of MST1 on the subcellular localization of FOXO3, CCL39 cells, which are optimal for localization studies of FOXO3 were employed. MST1, but not MST1 K59R, stimulated the accumulation of GFP-FOXO3 in the nucleus of these cells (FIG. 10). However, MST 1 failed to induce the nuclear accumulation of the GFP-FOXO3 mutants in which serine 207 was replaced with alanine (FIG. 4E). In neurons, hydrogen peroxide induced the translocation of GFP-FOXO3 but not of the GFP-FOXO3 S207A mutant (FIG. 4F). These results indicate that the phosphorylation of FOXO3 at serine 207 mediates hydrogen peroxide-induced FOXO3 translocation to the nucleus. In control experiments, mutation of serine 207 had little effect on direct binding of FOXO3 to promoter of the FOXO3 target gene BIM (FIG. 11).

### MST-FOXO Signaling Mediates Oxidative Stress-Induced Transcription and Neuronal Death

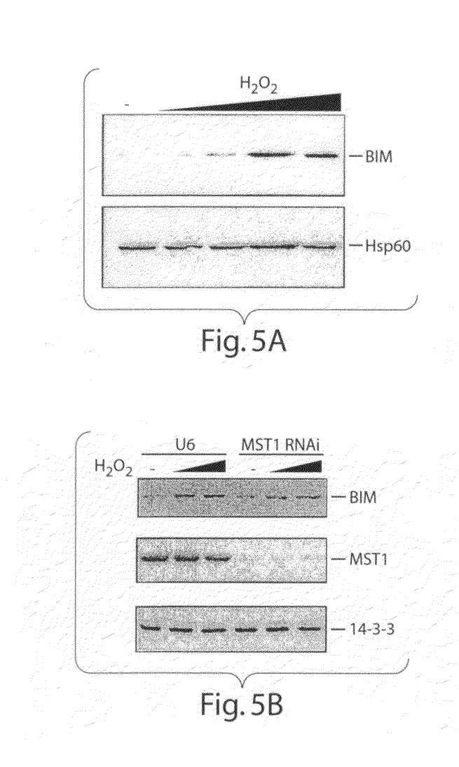

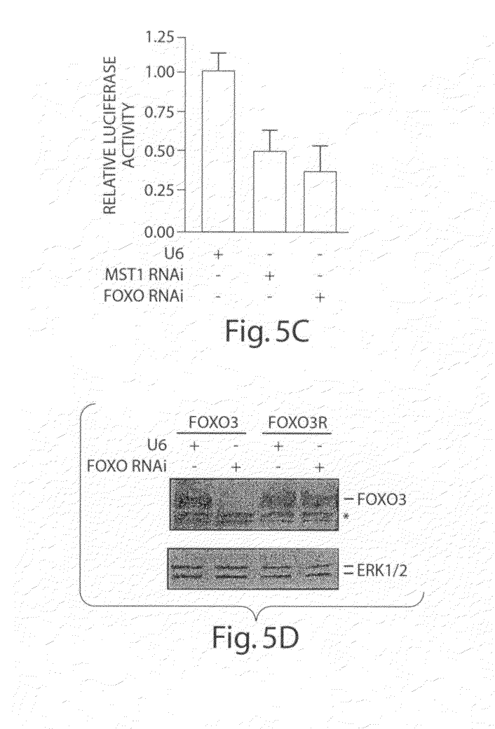

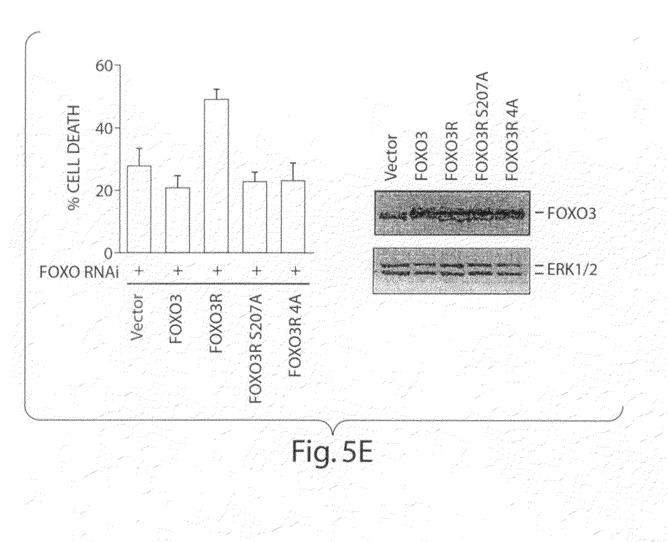

The identification of a signaling link between MST1 and FOXO3 that leads to the nuclear translocation of FOXO3 raised the possibility that the MST-FOXO pathway might couple oxidative stress signals to gene transcription and cell death. The FOXO3 target gene BIM encodes a BH3-only protein that directly activates the cell death machinery (Gilley et al., 2003). Hydrogen peroxide induced the expression of BIM protein in cells and neurons (FIGS. 5A and 5B). MST1 knockdown reduced the hydrogen peroxide-induced expression of BIM (FIG. 5B). In addition, MST1 and FOXO knockdown both significantly reduced BIM promoter-mediated transcription in hydrogen peroxide-treated granule neurons (FIG. 5C).

The significance of the MST1-induced FOXO3 phosphorylation in oxidative stress-induced neuronal cell death was next determined. Expression of an RNAi-resistant form of FOXO3 (FOXO3R), but not FOXO3 that is encoded by wild type cDNA, reversed the ability of FOXO RNAi to protect neurons from hydrogen peroxide-induced cell death (FIGS. 5D and 5E). In contrast to FOXO3R, FOXO3R mutants in which serine 207 was replaced with alanine (FOXO3R S207A or FOXO3R 4A) failed to mediate neuronal cell death in the background of FOXO RNAi (FIG. 5E). In control experiments, mutation of serine 207 had no effect on the amount of FOXO3R expression (FIG. 5E). These results indicate that phosphorylation of FOXO3 at serine 207 is required for the ability of FOXO3 to mediate hydrogen peroxide-induced neuronal cell death.

Taken together, our findings suggest that exposure of primary mammalian neurons to acute oxidative stress stimulates the activation of MST1 and its association with FOXO3 leading to the phosphorylation of FOXO3 at serine 207. These MST1-dependent events, which occur with rapid kinetics, in turn induce the dissociation of FOXO3 from 14-3-3 proteins and FOXO3 translocation to the nucleus culminating in neuronal cell death.

The MST-FOXO Signaling Pathway Promotes Longevity in C. elegans

The elucidation of the MST-FOXO signaling pathway in mammalian cells led to the determination of whether this signaling connection is conserved across species. The C. elegans model system has provided important insights into the functions and regulation of FOXO proteins (reviewed in Kenyon, 2005). The FOXO ortholog DAF-16 has not been implicated in the regulation of cell death, but DAF-16 is a central positive regulator of organismal longevity (Kenyon, 2005). DAF-16 function is inhibited by the C. elegans insulin/IGF1 receptor ortholog DAF-2 (Kenyon, 2005). The entire PI3K-Akt-FOXO signaling cascade is conserved in nematodes and mammals (Kenyon, 2005; Van Der Heide et al., 2004). Therefore, while inhibition of PI3K-Akt signaling triggers FOXO-dependent cell death in mammalian cells (Brunet et al., 2001; Van Der Heide et al., 2004), loss of function mutations in components of the DAF-2 signaling pathway, including daf-2, age-1, and akt, extend lifespan (Kenyon, 2005). Importantly, extension of lifespan by mutations of DAF-2 and other components of the DAF-2 pathway occurs in a DAF-16-dependent manner (Kenyon, 2005).

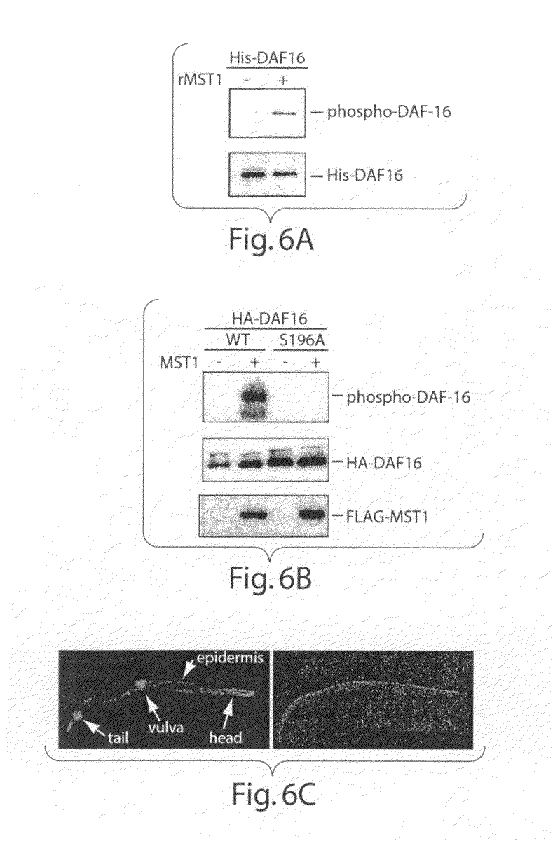

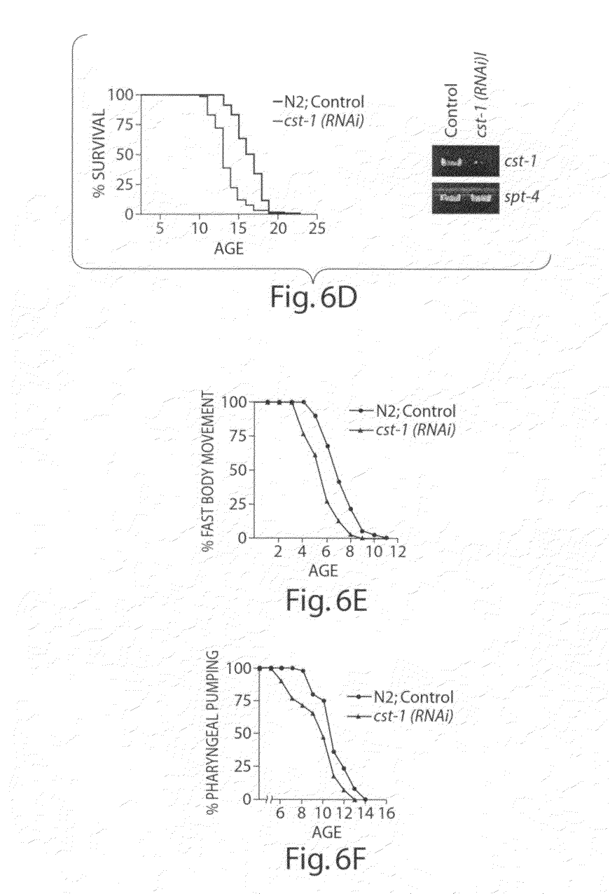

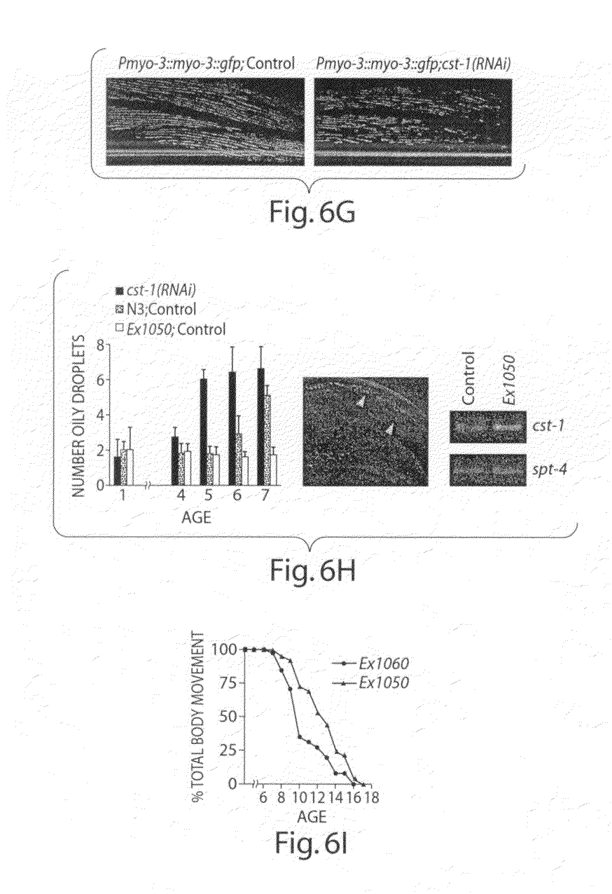

Since MST1-induced phosphorylation of FOXO3 activates its function in mammalian cells, MST orthologs in C. elegans would be predicted to promote DAF-16's ability to extend lifespan. To test this hypothesis, whether MST1 can phosphorylate DAF-16 at serine 196, which corresponds to serine 207 in FOXO3 (FIG. 2F) was next determined. MST1 robustly phosphorylated DAF-16 at the conserved forkhead site, serine 196, in vitro and in cells (FIGS. 6A and 6B). These results are consistent with the possibility that a nematode ortholog of MST1 might promote DAF-16 function in C. elegans and thus extend lifespan.

C. elegans contains two closely related genes that appear to represent orthologs of MST, cst-1 and cst-2 (C. elegans Ste20-like kinases 1 and 2) (FIGS. 12A and 12B). Transgenic nematodes that express GFP under the predicted cst-1 promoter (Pcst1::gfp) were generated. In five independent transgenic lines, Pcst1::gfp was widely expressed in epidermal cells and was accompanied by intense staining in the tail, vulva, and sensory neurons in the head and weaker expression in the dorsal pharyngeal bulb (FIG. 6C).

To assess the function of the CST kinases in aging in C. elegans, st-1 RNAi was induced by feeding in adult C. elegans. Reduction of cst-1 expression was confirmed by RT-PCR (FIG. 6D). Based on the high degree of homology of cst-1 and cst-2, the dsRNA encoded by the cst-1 RNAi construct is expected to target products of both cst genes (FIG. 12B); therefore, the effect of the RNAi construct is referred to as CST knockdown. CST knockdown by feeding RNAi had no obvious effects on nematode development, but it significantly shortened lifespan (FIG. 6D and Table 2).

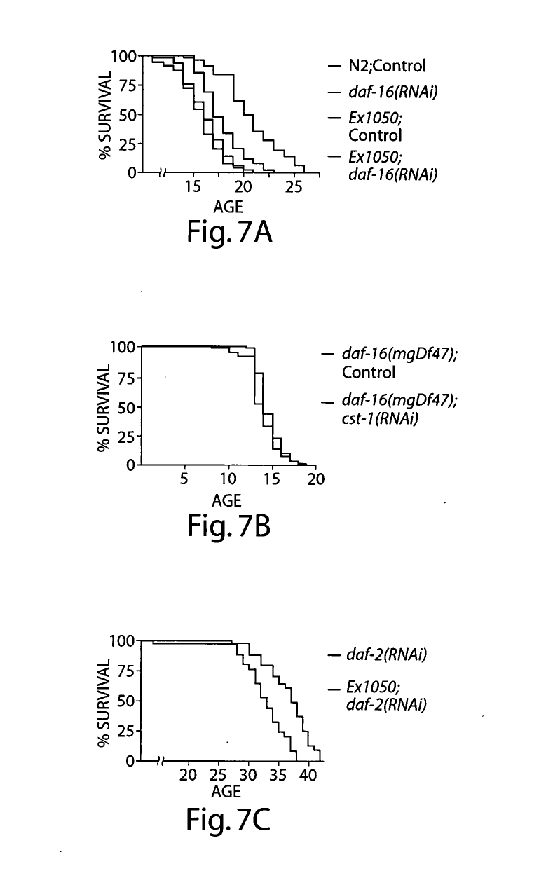

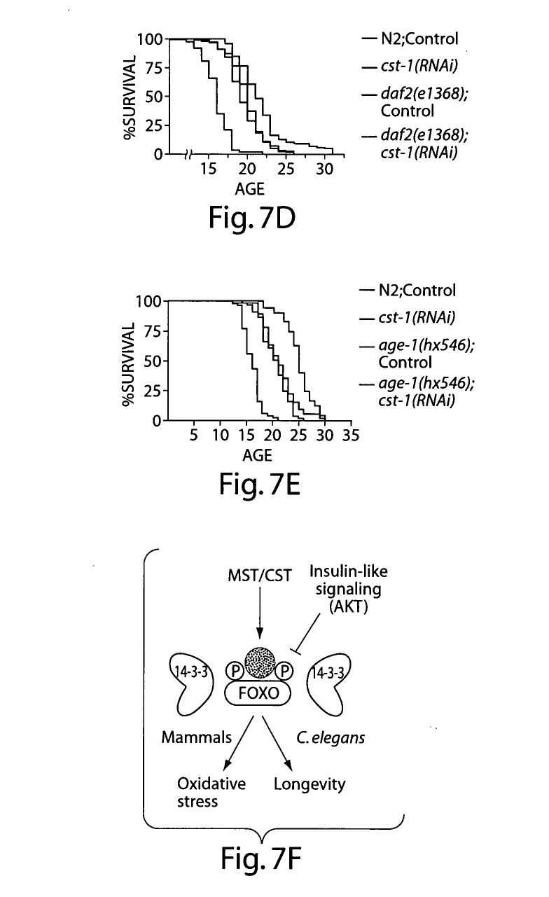

Reduction of lifespan upon gene knockdown may not reflect a specific effect on longevity. Therefore, to determine if CST specifically regulates lifespan, the expression of cst-1 in C. elegans was increased to determine its effect on lifespan. C. elegans carrying additional copies of the genomic locus of the cst-1 gene were generated. Increasing expression of the cst-1 gene in C. elegans significantly increased lifespan including the mean and 75th percentile lifespan (Table 2 and FIG. 7A). An increase in maximal lifespan upon cst-1 overexpression was also found (experiment 1: 32.3±0.7 days compared to N2 nematodes, 27.3±1.5 days; 2: 31.5±0.5 days compared to N2, 29.3±0.9 days; 3: 24.0±1.0 days compared to N2, 21.5±0.5 days; 4: 28.0±0.6 days compared to N2, 25.0±0.6 days; 5: 25.7±0.9 days compared to N2, 26.0±0.0 days; 6: 25.5±0.5 days compared to N2, 22.5±0.5 days; total number of animals observed/total initial number examined: Ex1050=479/493; N2=454/513). Thus, in five out of six experiments, maximal lifespan was significantly increased upon cst-1 overexpression as compared to N2 nematodes (t-test, p<0.05 in each of the five experiments). These results indicate that CST-1 promotes lifespan throughout the life of nematodes. Expression of the marker rol-6 for identification of transgenic nematodes alone (Ex1060) did not alter lifespan. Together, these findings indicate that CST-1 extends lifespan.

TABLE 2
Statistical analysis of adult nematode lifespan

| | | 75^{th} | Number of | |
|---|---|---|---|---|
| | Mean ± s.e.m. | percentile | animals that | |
| Background/Treatment | (days) | (days)* | died/total§ | p value# |
| Lifespans of N2 animals grown on | | | | |
| bacteria containing: | | | | |
| Control | 16.3 ± 0.2 | 18 | 97/100 | |
| cst-1 (RNAi) | 13.4 ± 0.2 | 14 | 96/100 | <0.0001∝ |
| Lifespans of daf-16 (mgDf47) | | | | |
| animals grown on bacteria | | | | |
| containing: | | | | |
| Control | 14.6 ± 0.1 | 15 | 93/100 | |
| cst-1 (RNAi) | 13.9 ± 0.2 | 15 | 98/100 | 0.0233∝ |
| Lifespans of N2 or daf-2 (e1368) | | | | |
| animals grown on bacteria | | | | |
| containing: | | | | |
| Control | 19.3 ± 0.2 | 21 | 180/209 | |
| cst-1 (RNAi) | 15.7 ± 0.1 | 17 | 182/209 | <0.0001∝ |
| daf-2 (e1368); Control | 23.8 ± 0.4 | 27 | 177/214 | |
| daf-2 (e1368); cst-1 (RNAi) | 20.7 ± 0.3 | 22 | 179/210 | <0.0001∝ |
| Lifespans of N2 or age-1 (hx546) | | | | |
| animals grown on bacteria | | | | |
| containing: | | | | |
| Control | 20.4 ± 0.4 | 22 | 58/60 | |
| cst-1 (RNAi) | 16.0 ± 0.3 | 17 | 51/61 | <0.0001∝ |
| age-1 (hx546); Control | 24.8 ± 0.4 | 26 | 50/59 | |
| age-1 (hx546); cst-1 (RNAi) | 21.1 ± 0.5 | 23 | 54/90 | <0.0001∝ |
| Lifespans of animals grown on | | | | |
| OP50: | | | | |
| N2 (control) | 18.8 ± 0.3 | 21 | 196/236 | |
| Ex1050 [Pcst-1::cst-1] | 20.9 ± 0.3 | 23 | 228/233 | <0.0001ζ |
| N2 (control) | 19.2 ± 0.3 | 21 | 92/115 | |
| Ex1051 [Pcst-1::cst-1] | 21.5 ± 0.4 | 24 | 87/97 | <0.0001ζ |
| Ex1052 [Pcst-1::cst-1] | 20.7 ± 0.3 | 22 | 106/121 | <0.005ζ |
| Lifespans of N2 or Ex1050 animals | | | | |
| grown on: | | | | |
| Control | 18.6 ± 0.2 | 20 | 258/277 | |
| daf-16 (RNAi) | 15.9 ± 0.1 | 17 | 246/259 | <0.0001θ |
| Ex1050; Control | 21.0 ± 0.2 | 23 | 251/260 | <0.0001θ |
| Ex1050; daf-16 (RNAi) | 16.2 ± 0.1 | 18 | 240/247 | 0.0250ω |
| Lifespans of N2 or Ex1050 animals | | | | |
| grown on: | | | | |
| daf-2 (RNAi) | 32.7 ± 0.4 | 35 | 75/75 | |
| Ex1050; daf-2 (RNAi) | 36.9 ± 0.6 | 40 | 67/68 | <0.0001φ |
| Lifespans of N2 or Ex1060 animals | | | | |
| grown on OP50: | | | | |
| N2 | 18.9 ± 0.3 | 21 | 46/74 | |
| Ex1060 [pRF4(rol-6(su1006))] | 19.2 ± 0.4 | 21 | 56/71 | 0.5541ζ |

*The 75^{th} percentile indicates the time point at which 25% of the nematodes were alive.
§Animals that crawled off the plate or exploded were censored from the data set. Total number of animals equals the total number of nematodes that died and the number censored.
#We used JMP-IN 5.1 statistical software for statistical analysis. p values are determined by log rank (Mantel-Cox) statistics. In the last column, p values with corresponding symbols are compared:
∝Compared with nematodes of identical genetic background cultured on HT115(DE3) bacteria containing control vector pL4440 plasmid.
ζCompared with N2 nematodes cultured on OP50 bacteria.
θCompared with N2 nematodes cultured on HT115(DE3) bacteria containing control vector pL4440 plasmid.
ωCompared with N2 nematodes cultured on HT115(DE3) bacteria containing daf-16 RNAi plasmid.
φCompared with N2 nematodes cultured on HT115(DE3) bacteria containing daf-2 RNAi plasmid.

Detailed analyses of age-associated physiological and pathological parameters after CST knockdown or overexpression were carried out. CST knockdown led to reduced body movement and pharyngeal pumping at an earlier age than N2 nematodes (FIGS. 6E and 6F) (Huang et al., 2004). Whether knockdown induces pathological features of aging muscle in nematodes was determined. As described for aging C. elegans (Herndon et al., 2002), sarcomeres of aging CST knockdown nematodes appeared more disorganized, containing discontinuities and bends in muscle fibers, when compared to control nematodes (FIG. 6G). CST knockdown also led to the premature appearance of age-associated oily droplets when compared to control N2 nematodes (FIG. 6H) (Herndon et al., 2002). These results indicate that CST knockdown accelerates tissue aging together with reducing lifespan.

Importantly, overexpression of cst-1 in C. elegans delayed the appearance of physical markers of aging as compared to control nematodes. Nematodes in which cst-1 was overexpressed (Ex1050) demonstrated a delay in the development of age-associated oily droplets (FIG. 6H), and were active for a longer period of time than control nematodes (FIG. 6I). Together, our results suggest that CST-1 specifically promotes longevity and delays aging.

The involvement of DAF-16 in CST-1-induced increase in nematode lifespan was determined. The effect of cst-1 overexpression on nematode lifespan in the background of daf-16 RNAi was assessed. Induction of DAF-16 knockdown shortened lifespan (FIG. 7A and Table 2). While cst-1 overexpression extended lifespan, daf-16 RNAi completely suppressed the ability of CST-1 to extend lifespan (FIG. 7A and Table 2). In other experiments, CST knockdown failed to reduce lifespan of DAF-16-deficient (daf-16-[mgDf47]) nematodes (FIG. 7B and Table 2). Taken together, these results suggest that DAF-16 acts downstream of CST-1 in lifespan extension.

The relationship of CST-1 and the insulin signaling pathway in lifespan control was analysed. Overexpression of cst-1 increased lifespan of daf-2 RNAi nematodes (FIG. 7C and Table 2). In addition, CST knockdown reduced lifespan to a similar extent in both N2 and DAF-2-deficient daf-2 (e1368) C. elegans as well as in both age-1 (hx546) and N2 nematodes (FIGS. 7D, 7E, and Table 2) (Gems et al., 1998; Friedman and Johnson, 1988). Since the daf-2 (e1368) and age-1 (hx546) alleles are non-null, epistasis between these genes and cst-1 cannot be definitively determined. However, taken together, our findings indicate that CST functions in parallel with the DAF-2 signaling pathway, converging on DAF-16 to regulate lifespan (see model in FIG. 7F).

MST family of kinases and the FOXO transcription factors that mediates responses to oxidative stress in mammalian cells and promotes longevity in nematodes (FIG. 7F). The identification of the FOXO proteins as major targets of MST indicate that these protein kinases play key roles in diverse biological processes including cellular homeostasis and longevity. Our genetic experiments in C. elegans indicate that MST activates FOXO function in both mammalian cells and nematodes. While MST1-induced phosphorylation of FOXO3 activates FOXO3-dependent cell death in mammalian neurons in response to oxidative stress, the nematode MST1 ortholog CST-1 activates DAF-16 function and thus promotes lifespan extension and delays aging of nematodes in a DAF-16-dependent manner.

Our study implicates the MST family of kinases as a key mediator of cellular responses to oxidative stress in higher eukaryotes. Oxidative stress in mammalian cells induces the MST1-mediated phosphorylation of FOXO3 at serine 207 leading to the release of FOXO3 from 14-3-3 proteins and consequent accumulation of FOXO3 in the nucleus, where FOXO3 induces expression of cell death genes (FIG. 7F). Activation of FOXO-dependent transcription can lead to either cell death or cell recovery in response to oxidative stress depending on the severity of the stimulus (Brunet et al., 2004; Essers et al., 2004). The participation of FOXO proteins may serve to expand the range of MST-induced cellular responses accompanying oxidative stress to include adaptive responses.

A key conclusion of our study is that the MST-FOXO signaling link is conserved. The characterization of the C. elegans ortholog CST-1 broadens MST functions beyond the control of cell death to the regulation of lifespan. In nematodes, the FOXO protein DAF-16 does not appear to have functions in cell death and instead plays a central role in longevity (Kenyon, 2005). Thus, CST-1 activation of DAF-16 provides a positive signal for lifespan extension. DAF-16 promotes lifespan regulation via a complex array of changes in gene expression (Lee et al., 2003a; McElwee et al., 2004; Murphy et al., 2003) In our experiments, cst-1 overexpression in nematodes induced the expression of the DAF-16 target gene hsp-12.6 (FIG. 13), which has been implicated in DAF-16-dependent lifespan extension (Hsu et al., 2003).

Long-lived nematodes carrying mutations of components of the DAF-2 signaling pathway are resistant to stress signals including reactive oxygen species (Honda and Honda, 2002). Consistent with these results, antioxidant and other stress-response genes constitute a prominent set of DAF-16-regulated genes (Lee et al., 2003a; McElwee et al., 2004; Murphy et al., 2003). However, the role of antioxidant genes including the widely studied gene superoxide dismutase sod-3 in lifespan regulation in C. elegans has not been established (Landis and Tower, 2005). Recent studies also point to several circumstances that uncouple lifespan extension from resistance to stress signals (Lee et al., 2003b; Libina et al., 2003). In view of these observations, further studies of the CST-DAF-16 pathway are required to shed light on the role of stress signals in longevity.

The elucidation of the MST1-induced phosphorylation of FOXO proteins and consequent disruption of their interaction with 14-3-3 proteins provides a molecular basis for how MST kinases activate FOXO signaling in both contexts of responses to oxidative stress in mammalian cells and the promotion of longevity in nematodes (FIG. 7F). Growth factors induce the Akt-mediated phosphorylation of FOXOs at distinct sites stimulating interaction with 14-3-3 proteins, which in turn both promote the nuclear export and inhibit the nuclear import of FOXO proteins (reviewed in Van Der Heide et al., 2004). The MST1-induced phosphorylation of FOXO proteins disrupts their interaction with 14-3-3 proteins and consequently promotes the nuclear accumulation of FOXOs. These observations indicate that MST/CST-induced phosphorylation of FOXO/DAF-16 opposes growth factor-regulation of FOXO transcription factors. Thus, the MST signal is a novel modulator of the insulin/IGF1-regulated PI3K-Akt-FOXO signaling pathway at the level of FOXO proteins.

Recent evidence suggests that the protein kinase JNK also signals via FOXO proteins to trigger cellular and organism-wide responses by opposing growth factor-regulation of FOXO proteins (Essers et al., 2004; Oh et al., 2005; Wang et al., 2005). JNK phosphorylates FOXO4 within the transactivation domain (Essers et al., 2004). In our experiments, JNK failed to phosphorylate the forkhead domain of FOXO proteins in vitro indicating that MST1 phosphorylates the conserved FOXO forkhead domain site independently of JNK. Together, these observations indicate that MST1 and JNK cooperate in the activation of FOXO proteins, whereby MST1 triggers the translocation of FOXOs to the nucleus to set the stage for JNK-induced phosphorylation of the FOXO transactivation domain.

The present study is based on the finding that the MST-FOXO signaling pathway is involved in responses to oxidative stress in mammalian cells and lifespan control in nematodes. However, identification of the signaling link between the MST kinases and the FOXO transcription factors points to new biological roles for both families of proteins. Since the FOXO transcription factors influence cell metabolism, differentiation, and transformation (Accili and Arden, 2004), our findings also indicate that MST might play important roles in these fundamental biological processes. In a similar vein, the role of FOXOs in pathological states including cancer and diabetes mellitus (Hu et al., 2004; Nakae et al., 2002) indicate that misregulation of MST-FOXO signaling contribute to the pathogenesis of these disorders. Finally, elucidation of the MST-FOXO signaling pathway as a key mediator of oxidative stress-induced neuronal cell death indicate that activation of this signaling pathway contributes to the pathogenesis of neurologic diseases.

What is claimed is:

1. A method of preferentially reducing or preventing neuronal cell death by contacting said cell with an agent that reduces the level or activity of an MST protein.

2. The method of claim 1, wherein said MST protein is an MST1 or MST2 protein.

3. The method of claim 1, wherein said agent reduces the level or activity of a FOXO transcription factor.

4. The method of claim 3, wherein said FOXO transcription factor is FOXO3.

5. The method of claim 1, wherein said agent is a small molecule inhibitor or an RNA interfering molecule.

6. A method of treating or preventing a neurologic disorder by administering to a mammal an agent that reduces the level or activity of an MST protein.

7. The method of claim 6, wherein said neurologic disorder is Alzheimer's disease, multiple sclerosis, Parkinson's disease, amyotrophic lateral sclerosis, stroke, cerebral ischemic disease, Huntington's disease, spinal muscular atrophy, stroke, brain trauma, spinal cord injury, or diabetic neuropathy.

8. The method of claim 6, wherein said MST protein is an MST1 or MST2 protein.

9. The method of claim 6, wherein said agent reduces the level or activity of a FOXO transcription factor.

10. The method of claim 9, wherein said FOXO transcription factor is FOXO3.

11. The method of claim 6, wherein said agent is a small molecule inhibitor or an RNA interfering molecule.

12. The method of claim 6, wherein said mammal is further administered a second therapeutic regimen.

13. A method for identifying a candidate compound for reducing neural cell apoptosis, said method comprising: (a) contacting a cell expressing a MST1 gene with a candidate compound and (b) measuring MST1 gene expression or protein activity in said cell, wherein a reduction in the level of said expression or said activity in the presence of said compound compared to that in the absence of said compound indicates that said compound reduces neural cell apoptosis.

14. The method of claim 13, wherein said candidate compound reduces the level or activity of a FOXO transcription factor.

15. The method of claim 14, wherein said FOXO transcription factor is FOXO3.

16. The method of claim 13, wherein said MST1 gene is an MST1 fusion gene.

17. The method of claim 13, wherein step (b) comprises measuring expression of MST1 mRNA or protein.

18. The method of claim 13, wherein said cell is a mammalian cell.

19. The method of claim 18, wherein said cell is a rodent or human cell.

20. The method of claim 18, wherein said cell is a neural cell.

21. A method for identifying a candidate compound for reducing neural cell apoptosis, said method comprising: (a) contacting an MST1 protein with a candidate compound; and (b) determining whether said candidate compound binds to said MST1 protein, wherein binding of said compound to said MST1 protein indicates that said candidate compound reduces apoptosis.

22. The method of claim 21, wherein said agent reduces binding of MST1 to a FOXO transcription factor.

23. The method of claim 21, wherein said MST1 protein is human MST1 protein.

24. A method for identifying a candidate compound for reducing neural cell death, said method comprising: (a) contacting an MST1 protein with a candidate compound; and (b) determining whether said candidate compound reduces binding of MST1 to a FOXO transcription factor wherein a reduction in MST1/FOXO binding indicates that said compound reduces neural cell death.

25. The method of claim 24, wherein said MST1 protein is human MST1 protein.

26. A method of treating or preventing a disorder that involves oxidative stress by administering to a mammal an agent that reduces the level or activity of an MST protein, wherein said disorder is diabetic retinopathy, diabetic nephropathy, ischemic heart disease, peripheral vascular disease, or cancer.
